SEQUENCE LISTING (26 sequences)

SEQ ID NO: 1 - DNA, 439 bp, Candidatus Saccharibacteria genomosp. TM7-H1
gatgaacgctggcggcatgcctaacacatgcaagtcgatcggtaaggcccttcgggtacacgagaggcggacggctgagtaacgcgtgggaacgcaccctacactgagggataagataccgaaaggtgttctaataccgcatacggtcttcggattaaagtcttcggacggtgaaggagcggcccgcgtcatattaggtagttggtggggtaatggcctaccaagccgatgatgtgtagctggtctgagaggatgatcagccagactggaactgagaacggtccagactcctacgggaggcagcagtgaggaatattccacaatgggcgaaagcctgatggagcaatgccgcgtgcaggatgaaggccctcgggtcgtaaactgcttttaagagaagaatatgacggtaactaatgaataagggacggctaactacgtg

SEQ ID NO: 2 - DNA, 480 bp, Clostridiales genomosp. BVAB1
gatgaacgttggcggcgtgcttaacacatgcaagtcgaacgaagtgctacgacggaagttttcggacggaagatgtagttacttagtggcggacgggtgagtaacgcgtggggaacctgccctgtaccgggggatagcagccggaaacggctgataataccgcataagcgcacaaatgtcgcatgacatggtgtgaaaaactccggtggtataggatggacccgcgtctgattagccggttggtggggtaaaagcctaccaaagcgaagatcagtagccgagttgagagactgaccggccacattgggactgagacacggcccagactcctacgggaggcagcagtggggaatattgcacaatgggcgaaagcctgatgcagcgacgccgcgtgagcgaagaagtatttcggtatgtaaagctctatcagaagggaagaaaatgacggtaccttactaagaagctccggctaaatacgtg

SEQ ID NO: 3 - DNA, 463 bp, Sneathia amnii
gataaacgctgacagaatgcttaacacatgcaagtctatgaggaagtttagcttgctaaatggactcatggcggacgggtgagtaacgcgtaaagaacttgccctttagactgggataacagagggaaacttctgataatactggataagttagtatatcgcatgatatgcaaatgaaagctacggcactaaaggagagctttgcgtcctattagctagttggtaaggtaagagcttaccaaggcgatgataggtagccggcctgagagggtggacggccacaaggggactgagatacggcccttactcctacgggaggcagcagtggggaatattggacaatggagggaactctgatccagcaattctgtgtgtgtgaagaaggttttaggactgtaaaacacttttagtagggaagaaaaaaatgacggtacctacagaagaagcaacggctaaatacgtg

SEQ ID NO: 4 - DNA, 490 bp, Prevotella OTU46
gatgaacgctagctacaggcttaacacatgcaagtcgcagggtaacgtgagggaagcttgctttccttgacgacgactggcgcacgggtgagtaacgcgtatccaaccttcccatgaccacgggataacccgttgaaagacggcctaataccgtatgacgtcgtttgccgacatcgaaagacgaccaaaggtttggcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatgggcgagagcctgaaccagccaagtagcgtgcaggatgacggccctatgggttgtaaactgcttttacgcggggataaagtgcgtgacgtgtctcgcattgcaggtaccgcgtgaataaggaccggctaattccgtg

SEQ ID NO: 5 - DNA, 490 bp, Prevotella buccalis
gatgaacgctagctacaggcttaacacatgcaagtcgcagggtaacgtgagggaagcttgcttcccttgacgacgactggcgcacgggtgagtaacgcgtatccaaccttcccatgaccacgggataacccgttgaaagacggactaataccgtatgacgtcgtttgctgacatcaaataacgattaaaggtttagcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatgggcgagagcctgaaccagccaagtagcgtgcaggatgacggccctatgggttgtaaactgcttttatgcggggataaagtgcgcgacgtgtcgtgcattgcaggtaccgcatgaataaggaccggctaattccgtg

SEQ ID NO: 6 - DNA, 490 bp, Prevotella timonensis
gatgaacgctagctacaggcttaacacatgcaagtcgcagggtaacatgaggaaagcttgctttccttgatgacgactggcgcacgggtgagtaacgcgtatccaaccttcccataactacgggataacccgttgaaagacggcctaataccgtatgatatcgtttgctgacatcaaataacgattaaaggtttagcggttatggatggggatgcgtctgattagcttgttggcggggtaacggcccaccaaggctacgatcagtaggggttctgagaggaaggtcccccacattggaactgagacacggtccaaactcctacgggaggcagcagtgaggaatattggtcaatgggcgagagcctgaaccagccaagtagcgtgcaggatgacggccctatgggttgtaaactgcttttatgtggggataaagtgcgtgacgtgtcatgcattgcaggtaccacatgaataaggaccggctaattccgtg

SEQ ID NO: 7 - DNA, 490 bp, Prevotella OTU47
gatgaacgctagctacaggcttaacacatgcaagttgcagggtaacatgagagaagcttgcttttcttgatgacgactggcgcacgggtgagtaacgcgtatccaaccttcccataactacgggataacccgttgaaagacggcctaataccgtataacatcgtttgcagacatctaacgacgattaaaggtttagcggttatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatgggcgagagcctgaaccagccaagtagcgtgtaggatgacggccctatgggttgtaaactacttttgtgtggggataaagtgaggcacgtgtgccttattgcaggtaccacacgaataaggaccggctaattccgtg

SEQ ID NO: 8 - DNA, 514 bp, Dialister OTU30
gacgaacgctggcggcgtgcttaacacatgcaagtcgaacgggaagacatgaagagcttgctctttatgaaatccagtggcaaacgggtgagtaacacgtaaacaacctgccttcaggatggggacaacagacggaaacgactgctaataccgaatacgattcttgagtcgcatgacacaagaaagaaagggtggcctctacttgtaagctatcgcctggagaggggtttgcgtccgattaggtagttggtgaggtaacggcccaccaagccgacgatcggtagccggtctgagaggatgaacggccacattggaactgagacacggtccagactcctacgggaggcagcagtggggaatcttccgcaatgggcgaaagcctgacggagcaacgccgcgtgagtgaagacggcctcgggttgtaaactactgtgattcgggacgaaggataagtagacgaataatactgcataagtagacggtaccgaaaagacaagaccacggctaacacgtg

SEQ ID NO: 9 - DNA, 513 bp, Dialister OTU167
gacgaacgctggcggcgtgcttaacacatgcaagtcgaacgggaagacatgaagagcttgctctttatgaaatccagtggcaaacgggtgagtaacacgtaaacaacctgccttcaggatggggacaacagacggaaacgactgctaataccgaatacgattcttgagtcgcatgacacaagaaagaaagggtggcctctacaagtaagctatcgcctgaagaggggtttgcgtccgattaggtagttggtgaggtaacggcccaccaagccgacgatcggtagccggtctgagaggatgaacggccacattggaactgagacacggtccagactcctacgggaggcagcagtggggaatcttccgcaatgggcgaaagcctgacggagcaacgccgcgtgagtgaagacggccttcgggttgtaaaactctgtgattcgggacgaaagataagcagacgaataatctgcataagtgacggtaccgaaaaagcaagccacggctaactacgtg

SEQ ID NO: 10 - DNA, 490 bp, Prevotella amnii
gatgaacgctagctataggcttaacacatgcaagtcgaggggcagcatatagattgcttgcaatttatgatggcgaccggcgcacgggtgagtaacgcgtatccaacctacccattactagggaataacccagcgaaagttggcctaatgccctatgtagtcgtttgatcgcctgagatttcgacgaaagatttatcggtattggatggggatgcgtctgattagcttgttggcggggtaaaggcccaccaaggcgacgatcagtaggggttctgagaggaaggtcccccacattggaactgagacacggtccaaactcctacgggaggcagcagtgaggaatattggtcaatgggcgagagcctgaaccagccaagtagcgtgcaggatgacggccctatgggttgtaaactgcttttatatgggaataaagtgagggacgtgtcccttattgcatgtaccatatgaataaggaccggctaattccgtg

SEQ ID NO: 11 - DNA, 466 bp, Sneathia sanguinegens
gataaacgctgacagaatgcttaacacatgcaagtcgatgatgggagctagcttgctagaagaagtcatggcggacgggtgagtaacgtgtaaagaacttaccatatagactgggataacagagggaaacttctgataatactggataagttagtagtagcattactaagtaatgaaaggtagcaatacgctatatgagagctttgcatcctattagctagttggtggggtaaaagcctaccaaggcgatgataggtagccggcctgagagggtggacggccacaaggggactgagatacggcccttactcctacgggaggcagcagtggggaatattggacaatggaggcaactctgatccagcaattctgtgtgtgtgaagaaggttttaggactgtaaaacacattttagtagggaagaaagaaatgacggtacctacagaagaagcgacggctaaatacgtg

SEQ ID NO: 12 - DNA, 497 bp, Aerococcus christensenii
gacgaacgctggcggcgtgcctaatacatgcaagtcgagcgaacagagaaagtgcttgcactttcaaagttagcggcggacgggtgagtaacacgtaaggaacctaccgataagcgggggacaacatccggaaacgggtgctaataccgcataggaagtttgttcgcatgaacaaacttagaaagatggctctgctatcacttatcgatggccttgcggtgcattaactagttggcgaggtaacggctcaccaaggtgatgatgcatagccgacctgagagggtaatcggccacattgggactgagacacggcccaaactcctacgggaggcagcagtagggaatcttccgcaatggacgcaagtctgacggagcaacgccgcgtgagtgaagaaggttttcggatcgtaaaactctgttgtaagagaagaacaaattgtagagtaactgctacagtcttgacggtatcttaccagaaagccacggctaactacgtg

SEQ ID NO: 13 - DNA, 487 bp, Clostridales BVAB2
gatgaacgctggcggcgtgcttaacacatgcaagtcgaacggagttaatttgaggaagcaagcttgcttgaagaattaaattaacttagtggcggacgggcgagtaacacgtgagcaacctgcctcttacaggggaataacaacgggaaaccgttgctaataccgcataacatgttgaaagggcatccttttaacatcaaaggagcaatccggtaagagatgggctcgcgtccgattagctagttggtagggtaacggcctaccaaggcgacgatcggtagccggactgagaggtcgaacggccgcattgggactgagacacggcccagactcctacgggaggcagcagtggggaatattgggcaatgggcgaaagcctgacccagcaacgccgcgtgagtgatgaaggccttcgggttgtaaaactctttggacagggacgaagaaagtgacggtacctgtagaacaagccacggctaactacgtg

SEQ ID NO: 14 - DNA, 462 bp, Coriobacteriaceae OTU27
gatgaacgctggcggcgtgcctaacacatgcaagtcgaacgattaaagcaccttcgggtgtgtatagagtggcgaacgggtgagtaacacgtgaccaacctgcctcttacattgggacaaccaaaagaaattctggctaataccaaatactccgcacatatcacatgatgtatgcgggaaagcttttgcggtaagagatggggtcgcggcccattaggtagacggcggggtagaagcccaccgtgccgatgatgggtagccgggttgagagaccgaccggccacattgggactgagatacggcccagactcctacgggaggcagcagtggggaatattgcgcaatgggggaaaccctgacgcagcaacgccgcgtgcgggatgaaggccttcgggttgtaaaccgctttcagcagggaagacatcgacggtacctgcagaagaagccccggctaactacgtg

SEQ ID NO: 15 - DNA, 511 bp, Dialister micraerophilus
gacgaacgctggcggcgtgcttaacacatgcaagtcgaacgagaggacatgaaaagcttgctttttatgaaatctagtggcaaacgggtgagtaacacgtaaacaacctgccttcaagatggggacaacagacggaaacgactgctaataccgaatacgatccgaaagtcgcatgacatttggatgaaagggtggcctatcgaagaagctatcgcttgaagaggggtttgcgtccgattaggtagttggtgaggtaacggcccaccaagccgacgatcggtagccggtctgagaggatgaacggccacactggaactgagacacggtccagactcctacgggaggcagcagtggggaatcttccgcaatggacgaaagtctgacggagcaacgccgcgtgagtgaagacggccttcgggttgtaaagctctgtgattcgggacgaaaggccatatgtgaataatatatggaaatgacggtaccgaaaaagcaagccacggctaactacgtg

SEQ ID NO: 16 - DNA, 509 bp, Megasphaera OTU70 type 1
gacgaacgctggcggcgtgcttaacacatgcaagtcgaacgagaggacatgggaagcttgcttcctatgaaatcgagtggcaaacgggtgagtaacgcgtaaacaacctgcccttcggatggggataacagccggaaacggctgctaataccgaatacgatcttttcgtcgcatgacggaaagaagaaaggatggcctctatacaaagctatcgccgaaggaggggtttgcgtctgattagctggttggaggggtaacggcccaacaaggcgatgatcagtagccggtctgagaggatgaacggccacattgggactgagacacggcccagactcctacgggaggcagcagtggggaatcttccgcaatggacgaaagtctgacggagcaacgccgcgtgagtgaagaaggtcttcggattgtaaactctgttatacgggacgaaaagacggatgccaacagtatccgtccgtgacggtaccgtaagagaagccacggctaactacgtg

SEQ ID NO: 17 - DNA, 480 bp, Parvimonas OTU142
gacgaacgctggcggcgtgcttaacacatgcaagtcgaacgagagttagatcgaatgagttttcggacaagtgagatttaacgaaagtggcgaacgggtgagtaacacgtgagcaacctgccttacacagggggatagccattggaaacggtgattaataccccataagaccacaataccgcatggtaaaagggtaaaagggataccggtgtaagatgggctcgcgtctgattagctagttggtggggtaaaggcctaccaaggcgacgatcagtagccggtctgagaggatgaacggccacattggaactgagacacggtccaaactcctacgggaggcagcagtggggaatattgcacaatgggggaaaccctgatgcagcgacgccgcgtgagcgaagaaggctttcgagtcgtaaagctctgtcctatgagaagataatgacggtatcataggaggaagccctggctaaatacgtg

SEQ ID NO: 18 - DNA, 519 bp, Lactobacillus crispatus NCTC 4
gacgaacgctggcggcgtgcctaatacatgcaagtcgagcgagcttgcctagatgaatttggtgcttgcaccagatgaaactagatacaagcgagcggcggatgggtgagtaacacgtggggaacctgccccatagtctgggataccacttggaaacaggtgctaataccggataagaacactagatcgcatgatcagcttttaaaaggcggcgtaagctgtcgctatgggatggccccgcggtgcattagctagttggtaaggtaaaggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttcggctcgtaaagctctgttggtagtgaagaaggatagaggtagtaactggcctttatttgacggtaatcaaccagaaagtcacggctaactacgtg

SEQ ID NO: 19 - DNA, 512 bp, Lactobacillus crispatus strain ST1
gacgaacgctggcggcgtgcctaatacatgcaagtcgagcgagcggaactaacagatttacttcggtaatgacgttaggaaagcgagcggcggatgggtgagtaacacgtggggaacctgccccatagtctgggataccacttggaaacaggtgctaataccggataagaaagcagatcgcatgatcagcttttaaaaggcggcgtaagctgtcgctatgggatggccccgcggtgcattagctagttggtaaggtaaaggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

SEQ ID NO: 20 - DNA, 512 bp, Lactobacillus acidophilus
gacgaacgctggcggcgtgcctaatacatgcaagtcgagcgagctgaaccaacagattcacttcggtgatgacgttgggaacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtagggtaacggcctaccaaggcaatgatgcatagccgagttgagagactgatcggccacattgggactgagacacggcccaaactcctacgggaggcagcagtagggaatcttccacaatggacgaaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

SEQ ID NO: 21 - DNA, 512 bp, Lactobacillus amylovorus
gacgaacgctggcggcgtgcctaatacatgcaagtcgagcgagcggaaccaacagatttacttcggtaatgacgctgggaaagcgagcggcggatgggtgagtaacacgtggggaacctgcccctaagtctgggataccatttggaaacaggtgctaataccggataataaagcagatcgcatgatcagcttttgaaaggcggcgtaagctgtcgctaagggatggccccgcggtgcattagctagttggtaaggtaacggcttaccaaggcgacgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

SEQ ID NO: 22 - DNA, 512 bp, Lactobacillus gallinarum
gacgaacgctggcggcgtgcctaatacatgcaagtcgagcgagcagaaccagcagatttacttcggtaatgacgctggggacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtaaggtaacggcttaccaaggcgatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcagatcgtaaagctctgttgttggtgaagaaggatagaggtagtaactggcctttatttgacggtaatcaaccagaaagtcacggctaactacgtg

SEQ ID NO: 23 - DNA, 512 bp, Lactobacillus helveticus
gacgaacgctggcggcgtgcctaatacatgcaagtcgagcgagcagaaccagcagatttacttcggtaatgacgctggggacgcgagcggcggatgggtgagtaacacgtggggaacctgccccatagtctgggataccacttggaaacaggtgctaataccggataagaaagcagatcgcatgatcagcttataaaaggcggcgtaagctgtcgctatgggatggccccgcggtgcattagctagttggtaaggtaacggcttaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

SEQ ID NO: 24 - DNA, 512 bp, Lactobacillus kitasatonis
gacgaacgctggcggcgtgcctaatacatgcaagtcgagcgagcggaaccaacagatttacttcggtaatgacgttgggaaagcgagcggcggatgggtgagtaacacgtggggaacctgcccctaagtctgggataccatttggaaacaggtgctaataccggataagaaagcagatcgcatgatcagcttttaaaaggcggcgtaagctgtcgctaagggatggccccgcggtgcattagctagttggtaaggtaacggcttaccaaggcaacgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

SEQ ID NO: 25 - DNA, 512 bp, Lactobacillus sobrius
gacgaacgctggcggcgtgcctaatacatgcaagtcgagcgagcggaaccaacagatttacttcggtaatgacgttgggaaagcgagcggcggatgggtgagtaacacgtggggaacctgcccctaagtctgggataccatttggaaacaggtgctaataccggataataaagcagatcgcatgatcagcttttgaaaggcggcgtaagctgtcgctaagggatggccccgcggtgcattagctagttggtaaggtaacggcttaccaaggcgacgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

SEQ ID NO: 26 - DNA, 512 bp, Lactobacillus ultunensis
gacgaacgctggcggcgtgcctaatacatgcaagtcgagcgagcggaaccagcagatctgcttcggcagtgacgctgggaaagcgagcggcggatgggtgagtaacacgtggggaacctgccccaaagtctgggataccacttggaaacaggtgctaataccggataagaaagcagatcgcatgatcagcttttaaaaggcggcgtaagctgtcgctatgggatggccccgcggtgcattagctagttggtagagtaacggcctaccaaggcaatgatgcatagccgagttgagagactgatcggccacattgggactgagacacggcccaaactcctacgggaggcagcagtagggaatcttccacaatggacgcaagtctgatggagcaacgccgcgtgagtgaagaaggttttcggatcgtaaagctctgttgttggtgaagaaggatagaggtagtaactggcctttatttgacggtaatcaaccagaaagtcacggctaactacgtg

(19) United States
(12) Patent Application Publication
(10) Pub. No.: US 2022/0243247 A1
(43) Pub. Date: Aug. 4, 2022

(54) VAGINAL MICROBIOME MARKERS FOR PREDICTION OF PREVENTION OF PRETERM BIRTH AND OTHER ADVERSE PREGNANCY OUTCOMES

(71) Applicant: VIRGINIA COMMONWEALTH UNIVERSITY, Richmond, VA (US)

(72) Inventors: Tom ARODZ, Richmond, VA (US); Gregory BUCK, Richmond, VA (US); Kimberly K. JEFFERSON, Richmond, VA (US); Jerome STRAUSS, Richmond, VA (US); Jennifer FETTWEIS, Richmond, VA (US); Myrna SERRANO, Richmond, VA (US); Philippe GIRERD, Richmond, VA (US); Paul BROOKS, Richmond, VA (US); David EDWARDS, Richmond, VA (US)

(21) Appl. No.: 17/608,242

(22) PCT Filed: May 1, 2020
(86) PCT No.: PCT/US2020/030934

§ 371 Date: Nov. 2, 2021

Related U.S. Application Data
(60) Provisional application No. 62/842,724, filed on May 3, 2019.

(51) Int. Cl. C12Q 1/06; C12Q 1/689

(52) U.S. Cl. CPC C12Q 1/06; C12Q 2600/16; C12Q 2600/118; C12Q 1/689

(57) ABSTRACT

A method for determining the risk of an adverse pregnancy outcome for a woman is provided comprising the steps of measuring a level of TM7-H1 and optionally one or more of BVAB1, Sneathia amnii, and Prevotella cluster 2 in a vaginal sample obtained from the woman, and identifying the woman as having an increased risk for an adverse pregnancy outcome, or other adverse pregnancy outcomes, if the levels are increased compared to corresponding standard control levels. Methods for the prophylactic treatment of subjects identified as being at increased risk for an adverse pregnancy outcome are also provided.

### CROSS-REFERENCE TO RELATED APPLICATIONS

This application claims the benefit of U.S. Provisional Application No. 62/842,724, filed May 3, 2019, which is hereby incorporated herein by reference in its entirety.

### STATEMENT REGARDING FEDERALLY SPONSORED RESEARCH OR DEVELOPMENT

This invention was made with government support under grant number R01 HD080784 awarded by the National Institutes of Health (NIH). The government has certain rights in the invention.

### FIELD OF THE INVENTION

The invention is generally related to vaginal microbiome signatures that are indicative of a higher risk for adverse pregnancy outcomes and the use of such signatures for the prevention of adverse pregnancy outcomes such as preterm birth, miscarriage, or preeclampsia.

### BACKGROUND OF THE INVENTION

The incidence of preterm birth, with its significant societal costs, remains above 10% worldwide. Approximately 15 million preterm births defined as those that occur before 37 weeks of gestation occur annually worldwide¹. Preterm birth (PTB) remains the second most common cause of neonatal death across the globe, and the most common cause of infant mortality in countries with middle and high income economies^{1,2}. The consequences of PTB persist from early childhood³⁴ into adolescence⁵⁻⁷ and adulthood⁸⁻¹⁰. In the US, striking population differences with respect to PTB have existed for decades, with women of African ancestry having a substantially larger burden of risk than women of European ancestry. The estimated annual cost of PTB in the US alone was over S26.2 billion in 2005¹¹. Despite these statistics, there remains a paucity of effective strategies for predicting and preventing PTB.

Although twin studies have documented that maternal and fetal genetics play roles in determining the length of gestation, environmental factors, including the microbiome, are the most important contributors to PTB, particularly among women of African ancestry¹². Microbe-induced inflammation resulting from urinary tract infection, sexually transmitted diseases including trichomoniasis, or bacterial vaginosis is thought to be a cause of PTB¹³⁻¹⁷. Ascension of microbes from the lower reproductive tract to the placenta, fetal membranes and uterine cavity^{14,18}, and hematogenous spread of periodontal pathogens from the mouth¹⁹⁻²¹, have also been invoked to explain the more than 30% of PTBs that are associated with microbial etiologies.

In contrast to other body sites, where high diversity of the microbiota is generally associated with health, a homogeneous Lactobacillus-dominated microbiome has long been considered the hallmark of a healthy female reproductive tract²². A microbiome with higher species diversity is considered less healthy, particularly in women with bacterial vaginosis, which is characterized by dysbiosis and the presence of variety of bacterial anaerobes including, but not limited to, Gardnerella vaginalis, Atopobium vaginae, taxa of the genera Megasphaera, Mobiluncus, Prevotella, Sneathia, and of the order Clostridiales (BVAB1/2/3)²³⁻²⁶. However, more advanced technologies have revealed a more complex picture suggesting that a healthy vaginal microbiome can sometimes be characterized by a diverse microbiota²⁷⁻³⁵.

Several studies have examined the relationship of the vaginal microbiome and the outcome of pregnancy, including but not limited to PTB³⁶⁻⁴⁶. Collectively, these studies suggest that the composition of the microbiome of the female reproductive tract has a significant, population-specific, impact on PTB risk. However, as of yet, no consistent significant associations have emerged between specific microbial taxa or combinations thereof and PTB, as well as other adverse pregnancy outcomes.

### SUMMARY

The present disclosure describes significant harbingers of adverse pregnancy outcomes such as PTB early in pregnancy or prior to pregnancy. A high-resolution taxon-specific analyses revealed a significant association between PTB and the proportional abundance of TM7-H1, BVAB1 (recently renamed to Lachnovaginosum genomospecies), Sneathia amnii, and a specific group of Prevotella, among others. A genomic analysis of these taxa identified virulence factors, and metatranscriptomic analyses confirmed that genes encoding these factors are expressed in the vaginal environment. These taxa were also generally associated with elevated vaginal levels of inflammatory cytokines, suggesting that complex host-microbiome interactions likely contribute to PTB. The disclosure thus provides screening methods having a high degree of sensitivity and specificity for the prediction of PTB. These methods allow for the early intervention and prophylaxis of PTB.

An aspect of the disclosure provides a method for determining the risk of an adverse pregnancy outcome such as PTB for a woman, comprising measuring a level or abundance of TM7-H1 and optionally one or more of BVAB1, Sneathia amnii, and Prevotella cluster 2 in a vaginal sample obtained from the woman, and identifying the woman as having an increased risk for PTB if the levels are increased compared to corresponding standard control levels. In some embodiments, the level or abundance of each of TM7-H1, BVAB1, Sneathia amnii, and Prevotella cluster 2 is measured.

In some embodiments, the method further comprises measuring a level or abundance of one or more of Dialister cluster 51 (comprising Dialister OTU30 and Dialister OTU167), Prevotella amnii, Sneathia sanguinegens, Aerococcus christensenii, Clostridales BVAB2, Coriobacteriaceae OTU27, Dialister micraerophilus, Parvimonas OTU142, Megasphaera OTU70 type 1, and Lactobacillus crispatus cluster (comprising L. crispatus, L. acidophilus, L. amylovorus, L. gallinarum, L. helveticus, L. kitasatonis, L. sobrius and L. ultunensis).

In some embodiments, the vaginal sample is obtained between 6 and 24 weeks of gestation. In other embodiments, the samples are obtained between 1 and 6 weeks of gestation, or prior to pregnancy in women who are planning on getting pregnant.

Another aspect of the disclosure provides a method for determining the risk of PTB for a woman and administering at least one PTB prophylactic treatment to a woman identified as being at risk for PTB, comprising i) measuring a level or abundance of TM7-H1 and optionally one or more of BVAB1, Sneathia amnii, and Prevotella cluster 2 in a vaginal sample obtained from the woman; ii) identifying the woman as having an increased risk for PTB if the levels or abundances are increased compared to corresponding standard control levels; and iii) administering the at least one PTB prophylactic treatment to the woman who is identified as having an increased risk for PTB.

In some embodiments, the at least one PTB prophylactic treatment is selected from the group consisting of antenatal corticosteroids, antibiotics, tocolytics, progesterone, cerclage application, feminine hygiene protocols, products that modify the conditions of the female reproductive tract, prebiotics, probiotics, microbial or bacteriophage preparations, vaginal microbial transplants, and combinations thereof.

### BRIEF DESCRIPTION OF THE DRAWINGS

FIGS. 1A-B. Vagitypes of a) 90 women who delivered at term (≥39 wks gestation), and b) 45 women who delivered prematurely (<37 wks gestation). Profiles were generated as described in the Example from the earliest sample collected from each participant. The outer rings identify the 13 community states, or vagitypes, into which these microbiome profiles fall.

FIG. 2. Abundance of taxa significantly different in PTB and TB cohorts from FIG. 1. Box shows median and interquartile range, whiskers extend from minimum to maximum values within each cohort. Taxa abbreviations: Lcricl, Lactobacillus crispatus (comprising L. crispatus, L. acidophilus, L. amylovorus, L. gallinarum, L. helveticus, L. kitasatonis, L. sobrius and L. ultunensis); BVAB1, Lachnospiraceae BVAB1; Pcl2, Prevotella cluster 2 (comprising Prevotella buccalis, Prevotella timonensis, Prevotella OTU46 and Prevotella OTU47); Samn, Sneathia amnii; Dc151, Dialister cluster 51 (comprising Dialister OTU30 and Dialister OTU167); Pamn, Prevotella amnii; Ssan, Sneathia sanguinegens; Achr, Aerococcus christensenii; BVAB2, Clostridiales BVAB2; CO27, Coriobacteriaceae OTU27; Dmic, Dialister micraerophilus; P142, Parvimonas OTU142.

FIG. 3. Longitudinal generalized additive mixed effect model (GAMM) of vaginal microbiome composition during pregnancy. The model incorporates BMI, ancestry (African or European), pregnancy outcome (PTB, TB), a smoother for gestational age, and a random subject effect was used to longitudinally model log-transformed relative abundances of vaginally relevant taxa. Each panel plots log-transformed abundances of taxa throughout pregnancy and the plots are ordered based on the p-values for pregnancy outcome in the GAMM from highest (top left) to lowest (bottom right).

FIGS. 4A-B. Sparse Canonical Correlation Analysis (sCCA). Cytokine abundance in vaginal samples from women who experience TB (a) or PTB (b) were subjected to an integrative sCCA using log-transformed cytokine levels and log-transformed taxonomic profiling data. Triangles represent bacterial taxa and dots represent cytokines. Note that the component 1 axis for the term birth sCCA (left) has been reversed for effective visual comparison with preterm birth sCCA.

FIGS. 5A-C. Taxa that significantly differ in PTB and TB cohorts. The distributions of proportional abundance of taxa significantly differ in PTB (n=31) and TB (n=59) cohorts; the earliest sample available for each subject within the first 24 weeks of pregnancy was used for each subject. Abundance values below 0.001 were rounded down to 0. The taxa are: BVAB1: Lachnospiraceae BVAB1, Pcl2: Prevotella cluster 2, Mty1: Megasphaera OTU70 type1, Samn: Sneathia amnii, TM7: TM7-H1, Dc151: Dialister cluster51, Pamn: Prevotella amnii, BVAB2: Clostridiales BVAB2, Dmic: Dialister micraerophilus and P142: Parvimonas OTU142. These 10 taxa have p<0.05 to support a significant difference in proportional abundance between PTB and TB cohorts using a Mann-Whitney U test (two-sided) and the Benjamini-Hochberg correction procedure with a False Discovery Rate of 5%. (A) Boxes show median and interquartile range; whiskers extend from minimum to maximum values within each cohort. (B, C) Scatter plot of the PTB predictive score returned by the model (horizontal axis) plotted against gestational age at birth (vertical axis). Each point corresponds to a sample from a subject: left bars—PTB subjects (n=31), right bars—TB subjects (n=59). (C) Shows more detailed view of the region where majority (48 of 59) of TB samples are located.

### DETAILED DESCRIPTION

Embodiments of the disclosure provide methods for the prediction of an elevated risk for an adverse pregnancy outcome such as preterm birth. The screening tests have high sensitivity and high specificity, and permit therapeutic intervention to decrease the risk.

Adverse pregnancy outcomes include, but are not limited to, spontaneous miscarriage, spontaneous abortion, preeclampsia, low birth weight, stillbirth, preterm rupture of membranes (PROM), preterm premature rupture of membranes (PPROM), and chorioamnionitis.

The term “preterm birth” (PTB) may be used interchangeably with “preterm delivery” (PTD) and refers to childbirth that occurs before the end of the 37^{th} week of gestation.

In one embodiment, the disclosure provides methods for diagnosing patients at risk for PTB based on the presence or absence of and/or the relative abundance of particular taxa of microbes in the vagina. Such patients have a higher than average or higher than normal chance of delivering a baby before 37 weeks of gestation as compared to individuals who have different vaginal microbes, or different amounts of microbes, or different relative amounts of microbes. Early identification of such a propensity allows early intervention, e.g. by altering the identity and/or the relative abundance of vaginal microflora associated with, and possibly causing, PTB, so that progression to PTB may be avoided, or delayed, or the associated symptoms may be lessened.

In some embodiments, a patient being screened by the methods disclosed herein may be asymptomatic with respect to PTB and may not have been previously deemed to be susceptible to PTB, e.g. pregnant nulliparous women or pregnant primiparous or multiparous women with no history of preterm birth. In other embodiments, a patient may be asymptomatic with respect to PTB, but for some reason, may be deemed susceptible to PTB (e.g. due to a previous history of PTB), and the methods of the invention provide a way to predict whether or not this is likely to occur. In some embodiments, the patient may already exhibit overtly one or more symptoms of PTB, such as a shortening cervix or frequent uterine contractions. In some embodiments, the patient is not pregnant and is screened prior to pregnancy.

In some embodiments, the identification of particular microflora (e.g. of particular phyla, genera or species of microbe(s)) may allow targeted therapies directed against the microbe or microbes which are undesirable, and/or therapies which increase the amount of desirable microflora, e.g. those which compete with the undesirable microbes, and/or which supply activities or produce substances which are beneficial, especially with respect to PTB.

In order to practice the methods described herein, generally a sample of vaginal microflora is obtained from the patient by any method known to those of skill in the art. The sample may be obtained from a pregnant woman at any time prior to 37 weeks of gestation, e.g. prior to 35, 34, 33, 32, 31, 30, 29, 28, 27, 26, 25, or 24 weeks. In some embodiments, the sample is obtained between 6 and 24 weeks of gestation, inclusive. In some embodiments, the sample is obtained prior to 6 weeks of gestation. In other embodiments, the sample is obtained prior to pregnancy.

The term “vaginal sample,” as used herein, refers to any vaginal sample suitable for testing or assaying according to the methods of the present invention. One example of a vaginal sample can be referred to as a gynecological sample, such as a vaginal swab obtained according to the procedures accepted in the medical field. However, the term “vaginal sample” is not limited to vaginal swabs, but can also be used to describe discharge or mucus samples, a cervical mucus sample, a cervical swab sample, a tissue sample or cell samples, obtained, processed, transported and stored using various suitable procedures. For examples, the samples can be stored in suitable storage or transportation devices, refrigerated, frozen, desiccated, diluted, mixed with various additives, or mounted on slides. In some embodiments, the “sample” may in fact be a urine sample which in most cases provides an accurate proxy for the ‘vaginal sample”.

The sample is tested for the presence or absence of, and/or for the relative abundance of, at least one bacteria selected from the group consisting of (with exemplary genomic Genbank accession numbers provided in parentheses): BVAB1 (also known as Clostridiales genomosp. BVAB1; PQV000000000), Sneathia amnii (NZ_CP011280), TM7-H1 (also known as Candidatus Saccharibacteria genomosp. TM7-H1; CP026537), Prevotella cluster 2 (NZ_ADEF01000048; Prevotella timonensis-NBAX01000001; Prevotella sp. BV3P1 NZ_AWXC00000000; Prevotella buccalis PNGJ01000001), Dialister cluster 51 (Veillonellaceae bacterium DNF00626; NZ_KQ960816), Prevotella amnii (KQ960470), Sneathia sanguinegens (LOQF01000001), Aerococcus christensenii (CP014159), Clostridales BVAB2 (closest taxa available Clostridiales bacterium KA00274; KQ959578 [id 95%]), Coriobacteriaceae OTU27 (Coriobacteriales bacterium DNF00809; NZ_KQ959671), Dialister micraerophilus (GL878519), Megasphaera OTU70 type 1 (ADGP01000001.1 and NZ_AFIJ01000040.1), and Parvimonas OTU142 (closest taxa available Parvimonas sp. KA00067; NZ_KQ960143.1 [id 88%]) wherein an increase in any of these bacteria as compared to a control is indicative of a higher risk for PTB. Prevotella cluster 2 includes at least the following taxa: Prevotella buccalis, Prevotella timonensis, Prevotella OTU46 and Prevotella OTU47). In some embodiments, the sample is also tested for the presence or absence of, and/or for the relative abundance of Lactobacillus crispatus (comprising L. crispatus, L. acidophilus, L. amylovorus, L. gallinarum, L. helveticus, L. kitasatonis, L. sobrius and L. ultunensis) cluster wherein a decrease in abundance as compared to a control is indicative of a higher risk for PTB.

An exemplary gene sequence which encodes for the 16S rRNA of TM7-H1 is represented by SEQ ID NO: 1. An exemplary gene sequence which encodes for the 16S rRNA of BVAB1 is represented by SEQ ID NO: 2. An exemplary gene sequence which encodes for the 16S rRNA of Sneathia amnii is represented by SEQ ID NO: 3. An exemplary gene sequence which encodes for the 16S rRNA of Prevotella OTU46 is represented by SEQ ID NO: 4. An exemplary gene sequence which encodes for the 16S rRNA of Prevotella buccalis is represented by SEQ ID NO: 5. An exemplary gene sequence which encodes for the 16S rRNA of Prevotella timonensis is represented by SEQ ID NO: 6. An exemplary gene sequence which encodes for the 16S rRNA of Prevotella OTU47 is represented by SEQ ID NO: 7. An exemplary gene sequence which encodes for the 16S rRNA of Dialister OTU30 is represented by SEQ ID NO: 8. An exemplary gene sequence which encodes for the 16S rRNA of Dialister OTU167 is represented by SEQ ID NO: 9. An exemplary gene sequence which encodes for the 16S rRNA of Prevotella amnii is represented by SEQ ID NO: 10. An exemplary gene sequence which encodes for the 16S rRNA of Sneathia sanguinegens is represented by SEQ ID NO: 11. An exemplary gene sequence which encodes for the 16S rRNA of Aerococcus christensenii is represented by SEQ ID NO: 12. An exemplary gene sequence which encodes for the 16S rRNA of Clostridales BVAB2 is represented by SEQ ID NO: 13. An exemplary gene sequence which encodes for the 16S rRNA of Coriobacteriaceae OTU27 is represented by SEQ ID NO: 14. An exemplary gene sequence which encodes for the 16S rRNA of Dialister micraerophilus is represented by SEQ ID NO: 15. An exemplary gene sequence which encodes for the 16S rRNA of Megasphaera OTU70 type 1 is represented by SEQ ID NO: 16. An exemplary gene sequence which encodes for the 16S rRNA of Parvimonas OTU142 is represented by SEQ ID NO: 17. An exemplary gene sequence which encodes for the 16S rRNA of Lactobacillus crispatus strain NCTC4 is represented by SEQ ID NO: 18. An exemplary gene sequence which encodes for the 16S rRNA of Lactobacillus crispatus strain ST1 is represented by SEQ ID NO: 19. An exemplary gene sequence which encodes for the 16S rRNA of Lactobacillus acidophilus is represented by SEQ ID NO: 20. An exemplary gene sequence which encodes for the 16S rRNA of Lactobacillus amylovorus is represented by SEQ ID NO: 21. An exemplary gene sequence which encodes for the 16S rRNA of Lactobacillus gallinarum is represented by SEQ ID NO: 22. An exemplary gene sequence which encodes for the 16S rRNA of Lactobacillus helveticus is represented by SEQ ID NO: 23. An exemplary gene sequence which encodes for the 16S rRNA of Lactobacillus kitasatonis is represented by SEQ ID NO: 24. An exemplary gene sequence which encodes for the 16S rRNA of Lactobacillus sobrius is represented by SEQ ID NO: 25. An exemplary gene sequence which encodes for the 16S rRNA of Lactobacillus ultunensis is represented by SEQ ID NO: 26.

In some embodiments, the level of at least one bacteria in the sample is measured. In some embodiments, a plurality, i.e. 2 or more, is measured, e.g. at least 2, 3, 4, 5, 6, 7, 8, 9, 10, 11, 12, 13, 14 or more. It is contemplated that the levels of bacteria other than those described herein may be measured in addition to those described herein. In some embodiments, the relative amount of bacteria in the sample is measured and compared to the relative amount in a control sample.

As used herein, “increase” or “decrease” refers to increasing or lowering by, for example, at least about 5%, 10%, 20%, 30%, 40%, 50%, 60%, 70%, 80%, 90%, or more when compared to at least one type of standard or control.

Diagnostic methods or tests according to some embodiments use bacterial markers that have high specificity and/or high sensitivity. The terms “sensitivity” and “specificity” are used herein to refer to statistical measures of the performance of diagnostics tests. Sensitivity refers to a proportion of positive results which are correctly identified by a test. Specificity measures a proportion of the negative results that are correctly identified by a test. The term “high specificity” refers to specificity that is equal to or over 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The term “high sensitivity” refers to sensitivity that is equal to or over 80%, 81%, 82%, 83%, 84%, 85%, 86%, 87%, 88%, 89%, 90%, 91%, 92%, 93%, 94%, 95%, 96%, 97%, 98%, or 99%.

Negative control samples for use in establishing negative standards of women unlikely to experience PTB (e.g. a “normal” or “healthy” vaginal microbiome signature) may be obtained from one or more subjects not at risk for PTB, for example, pregnant women who have exceeded a gestational age of 37 weeks, women who have delivered a baby at full term (i.e., at greater than 37 weeks), or non-pregnant women. Alternatively, or in addition, positive control samples may be used, e.g. samples obtained from subjects who have experienced PTB and/or are know to be at risk of PTB, to establish positive standards for women likely to experience PTB (e.g. an “at risk” vaginal microbiome signature). Further, standards may be refined to include specific stratified categories, examples of which include “very high risk” (PTB will occur without intervention), “moderate risk” (PTB is likely without intervention), and “low risk” (PTB is possible and the patient should be monitored but does not necessarily need intervention at the time). The risk levels may also take into account one or more factors in addition to the microbial signature, such as age, ethnic background, socioeconomic level, overall health, previous health records (including prior births with or without occurrences of PTB), etc.

Detection of microbes may be done in any of a number of ways that are known to those of ordinary skill in the art, including but not limited to culturing the organism(s), conducting various analyses which are indicative of the presence of the microbe(s) of interest (e.g. by microscopy, using staining techniques, enzyme assays, antibody assays, etc.), or by sequencing of genetic material (DNA or RNA) using, e.g. NextGen or Xgen technology, qPCR, chip technology, and others. While any category (or categories) of nucleic acid(s) may be detected (usually amplified using, e.g. PCR techniques), particularly useful amplification strategies include the use of primers (e.g. universal primers) which amplify ribosomal RNA genes (rRNA) as is known in the art. Other useful technologies include metagenomic or metatranscriptomic sequencing, in which all the DNA or RNA, respectively, in a sample is sequenced and used for taxonomic classification and determinations of abundance or relative abundance.

It will be appreciated that determining the abundance of microbes may be affected by taking into account any feature of the microbiome. Thus, the abundance of microbes may be affected by taking into account the abundance at different phylogenetic levels; at the level of gene abundance; gene metabolic pathway abundances; sub-species strain identification; SNPs and insertions and deletions in specific bacterial regions; growth rates of bacteria, the diversity of the microbes of the microbiome, etc.

In some embodiments, determining a level or set of levels of one or more types of microbes or components or products thereof comprises determining a level, abundance or relative abundance, or set of levels, abundances, or relative abundances of one or more DNA or RNA sequences. In some embodiments, one or more DNA or RNA sequences comprises any DNA or RNA sequence that can be used to differentiate between different microbial types. In certain embodiments, one or more DNA or RNA sequences comprises 16S rRNA gene or 16S rRNA sequences. In certain embodiments, one or more DNA or RNA sequences comprises 18S rRNA gene or 18S rRNA sequences. In some embodiments, 1, 2, 3, 4, 5, 10, 15, 20, 25, 50, 100, 1,000, 5,000 or more sequences are amplified. In some embodiments one or more DNA or RNA sequences comprises metagenomic or metatranscriptomic sequences of all the DNA or RNA in a sample.

16S and 18S rRNA gene sequences encode small subunit components of prokaryotic and eukaryotic ribosomes respectively. rRNA genes are particularly useful in distinguishing between types of microbes because, although sequences of these genes differs between microbial species, the genes have highly conserved regions for primer binding. This specificity between conserved primer binding regions allows the rRNA genes of many different types of microbes to be amplified with a single set of primers and then to be distinguished by amplified sequences.

In some embodiments, in order to classify a microbe as belonging to a particular genus, family, order, class or phylum, it must comprise at least 70% sequence homology, at least 75% sequence homology, at least 80% sequence homology, at least 85% sequence homology, at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to a particular taxon.

In some embodiments, in order to classify a microbe as belonging to a particular species, it must comprise at least 90% sequence homology, at least 91% sequence homology, at least 92% sequence homology, at least 93% sequence homology, at least 94% sequence homology, at least 95% sequence homology, at least 96% sequence homology, at least 97% sequence homology, at least 98% sequence homology, at least 99% sequence homology to a reference microbe known to belong to the particular species.

Once a patient is identified being at an elevated risk of PTB, suitable clinical intervention can be undertaken to prophylactically treat PTB. Exemplary treatments include but are not limited to: eliminating or lessening microflora which are increased in patients with PTB (e.g. using antibiotics or other therapies), promoting or increasing microflora which are decreased in patients with PTB (e.g., using prebiotics, probiotics, or other therapies), antenatal corticosteroids, tocolytics, progesterone, cerclage application, products that modify the conditions of the female reproductive tract (e.g., soaps such as those produced by Summer's Eve®), genetically engineered microbial or bacteriophage preparations, vaginal microbial transplants (e.g., with one or more species known to be associated with or conducive to a healthy microbiome that is not associated with PTB), and combinations thereof. Examples of microbes suitable for vaginal microbial transplants include but are not limited to: Lactobacillus species, including but not limited to L. crispatus, L. jensenii, L. gasseri, L. acidophilus, Lactobacillus GG, Lactobacillus rhamnosus, other Lactobacillus taxa, Bifidobacterium bifidum or other Bifidobacterium taxa, or other bacterial taxa.

A pregnant patient or subject to be treated by any of the methods of the present disclosure can mean either a human or a non-human animal including, but not limited to dogs, horses, cats, rabbits, gerbils, hamsters, rodents, birds, aquatic mammals, cattle, pigs, camelids, and other zoological animals.

In some embodiments, the treatment is administered to the subject in a therapeutically effective amount. By a “therapeutically effective amount” is meant a sufficient amount of active agent to decrease the likelihood of PTB at a reasonable benefit/risk ratio applicable to any medical treatment. In some aspects, PTB is prevented. In other aspects, the gestational time is increased until the infant has a reasonable chance of survival outside the uterus, i.e. at least up to about 22 weeks, and preferably longer, such as up to about 23, 24, 25, 26, 27, 28, 29, 30, 31, 32, 33, 34, 35, 36, 37, 38, 39 or 40 weeks.

Embodiments of the disclosure also provide methods for monitoring the efficacy of a treatment protocol that is ostensibly treating PTB. This might be early in pregnancy, or in some cases in women who are anticipating or planning on getting pregnant. The method involves determining vaginal microbiome signatures of a patient who is or who is going to be pregnant, and who may be treated to prevent PTB. Multiple signatures are generally obtained and analyzed at suitable time intervals, e.g., just prior to treatment to establish a baseline, and then repeatedly every few days or weeks thereafter. Subsequent signatures are compared to suitable reference signatures and/or to one or more previous signatures from the patient. If subsequent signatures indicate that the patient's vaginal microfloral signature is improving (e.g., is more similar to that of control subjects as described herein) then the treatment may be continued without adjustment, or may be gradually decreased, and may even be discontinued. However, if no improvement is observed, or if a signature indicates a worsening of the condition, then the treatment protocol can be adjusted accordingly, e.g., more of a treatment agent may be administered, or a different and/or more drastic form of treatment may be implemented, etc. The microflora signature is thus used to assess treatment adequacy and treatment response.

Embodiments of the present disclosure also include kits for use in the screening of PTB risk. For instance, such kits may include primer sets for the detection, amplification and classification of the bacterial strains present in a test sample taken from a host. The primer sets in such kits may include taxon-specific primer sets for classification of the bacteria present in the test sample. For instance, a kit may include primers that would allow for identification and classification of bacterial strains as described herein that are present in the test sample, so that comparison of relative amounts of bacteria present in the test sample can be determined and compared to a predetermined standard. Alternative kits may have home versions wherein antibodies, specific oligonucleotides, or other molecular methods for specific identification of a bacterial species, strain or taxon. These could be available as disposable dipsticks, akin to home pregnancy tests. Vaginal swabs, urine samples, or other appropriate specimens can be exposed to such dipsticks to detect and quantify bacterial taxa in the sample.

Before exemplary embodiments of the present invention are described in greater detail, it is to be understood that this invention is not limited to particular embodiments described, and as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

The invention is further described by the following non-limiting examples which further illustrate the invention, and are not intended, nor should they be interpreted to, limit the scope of the invention.

### Example

### Summary

Reported herein is an analysis of data generated in a collaborative effort under the umbrella of the National Institutes of Health's integrative Human Microbiome Project iHMP⁴⁷, representing a large cohort of pregnant women with deep characterization of the vaginal microbiome and risk of PTB. We identified differences in the vaginal microbial communities of women who experienced PTB, identified bacterial taxa associated with these differences, and showed that these bacteria express genes with pathogenic potential, supporting the concept that they elicit early induction of labor directly by infection, or indirectly by inducing inflammatory responses.

### Methods

### Participant Enrollment, Informed Consent, and Health History Collection

Participants for this study were enrolled from women visiting maternity clinics in Virginia and Seattle. All study procedures involving human subjects were reviewed and approved by the institutional review board at Virginia Commonwealth University (IRB #HM15527). Participants, approximately 1500, were enrolled from Women's Clinics at VCU Health Center, approximately 1000 women, and at multiple sites in Washington state, approximately 500 women, state by our partner registry, the Global Alliance to Prevent Prematurity and Stillbirth (GAPPS). Study protocols were harmonized across sites, and data and samples were all distributed to the VCU study for analysis. All study participants enrolled in Virginia and Washington were also enrolled in the Research Alliance for Microbiome Science (RAMS) Registry at Virginia Commonwealth University. RAMS Registry protocols were approved at Virginia Commonwealth University (IRB #HM15528); GAPPS-associated sites ceded review to the VCU IRB through reliance agreements. The study was performed with compliance to all relevant ethical regulations. Written informed consent was obtained from all participants and parental permission was obtained for participating minors.

Pregnant women and minors at least 15 years of age were provided literature on the project and invited to participate in the study. At initial visits, women over 18 or minors between 15 and 18 were administered informed consent or informed assent and parental permission, respectively. Women/minors who: 1) were incapable of understanding the informed consent or assent forms, or 2) were incarcerated were excluded from the study. Comprehensive demographic, health history and dietary assessment surveys were administered, and relevant clinical data (gestational age, height, weight, blood pressure, vaginal pH, diagnosis, etc.) was recorded. Relevant clinical information, e.g., gestational age, weight, any diagnosis, etc., was also obtained from neonates at birth and discharge.

At subsequent prenatal visits, triage, in labor and delivery, and at discharge, additional surveys were administered, relevant clinical data was recorded, and samples were collected. Samples included mid-vaginal, buccal, rectal, and skin swabs, urine, blood, cord, cord blood, placenta, membranes and amniotic fluid, baby buccal, baby rectal, baby skin, baby meconium and first stool. Vaginal and rectal samples were not collected at labor and delivery or at discharge. Women with any of the following conditions were excluded from sampling at a given visit:

1. Incapable of self-sampling due to mental, emotional or physical limitations.
2. More than minimal vaginal bleeding as judged by the clinician.
3. Ruptured membranes prior to 37 weeks.
4. Active herpes lesions in the vulvovaginal region.

### Case/Control Design

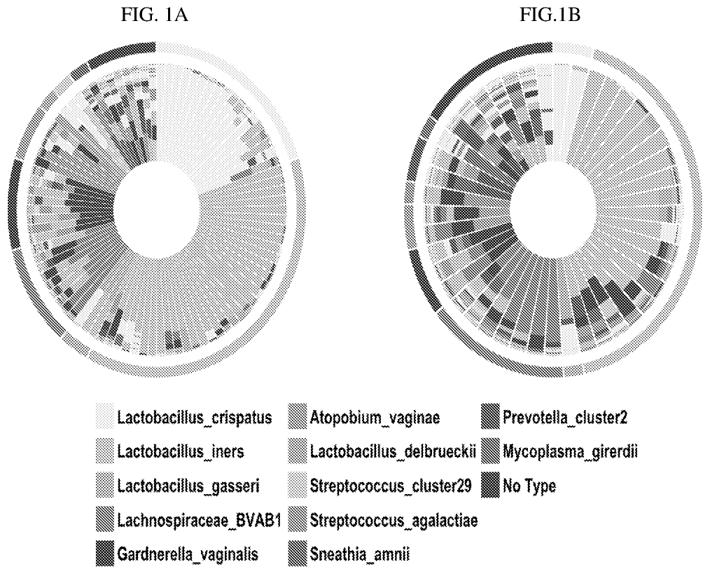

We selected 47 preterm cases of singleton, non-medically indicated preterm births from women who delivered between 20 and <37 weeks gestation from the women who enrolled in the Virginia arm of the study. The participants had completed the study through delivery, and their gestational age information had been recorded in the study operational database as of July 2016. We case-matched the preterm participants 2:1 with participants who completed the study with singleton term deliveries ≥39 weeks matching based on ethnicity, age and income. We matched cases as close to exact matches as possible. Most cases were matched using the in-house script; a few difficult-to-match cases were matched by hand. Case matching was performed blinded to all other study data. Two of the 47 preterm births did not have 16S rRNA that passes QC, thus these PTB samples and their controls were excluded from the taxonomic 16S rRNA analyses (FIG. 1) and demographic data in Table 1.

### Sample Collection

Samples (i.e., buccal, mid-vaginal wall, cervical, and rectal) were collected from appropriately consented women at the enrollment visit, longitudinally at each prenatal visit, at triage, and at labor and delivery. Samples were collected with BD BBL™ CultureSwab™ EZ swabs. Vaginal and rectal swabs were collected either by a clinician during a pelvic exam or by self-sampling. Cervical samples, when collected, were collected by a clinician during a pelvic exam using a speculum for the vaginal samples. Research coordinators collected buccal samples, and instructed the participants on self-sampling procedures, provided a self-sampling instructional brochure, and provided the participant a room for self-sampling. When samples were self-collected, no cervical samples were obtained. Self-sampling has been shown to provide samples equivalent to those collected by a trained clinician⁵⁸.

Samples were collected as follows: 1) mid-vaginal wall: a double-tipped CultureSwab™EZ swab was placed carefully on the vaginal sidewall about halfway between the introitus and the cervix, pressed firmly into the sidewall to a depth of roughly the diameter of the swab, and rolled dorsally-ventrally back and forth four times to coat the swab, and removed; 2) cervical: a single-tipped CultureSwab™EZ swab was inserted into the endocervix to the depth of the entire tip of the swab, rotated 360 degrees, held for ten seconds, and removed, being careful not to contact the vaginal walls; 3) buccal: a double-tipped CultureSwab™EZ was placed firmly in the mid-portion of the cheek and rubbed up and down 10 times while constantly rotating, and removed; 4) rectal: a CultureSwab™EZ swab was inserted ˜1 inch into rectum, rotated 360 degrees, held for ten seconds, and removed. Vaginal pH was collected using commercial applicators with pH paper. Briefly, the sterile applicators, miniature applicators with pH indicator affixed to the tip, were inserted ˜1.5-2 inches into vagina, applied gently to vaginal wall, and withdrawn. The pH was read by comparing the color of the pH indicator to a color chart by the Research Coordinator.

Samples from neonates (buccal, rectal, meconium and first stool) were collected at birth, at discharge, and in the NICU (if admitted). Buccal swabs were collected from neonates essentially as described above. Rectal swabs were collected using single-tip CultureSwab™EZ swab inserted ˜¼ inch into the rectum as described above. Meconium and first stool was collected from diapers using a sterile swab to collect a few grams of material.

### Sample Processing

After collection, swabs were immediately immersed in transfer buffer depending on the objectives; i.e., swabs for DNA isolation were immersed in MoBio PowerSoil® DNA Isolation buffer, swabs for RNA purification were immersed in RNAlater® (Qiagen), and swabs for cytokine profiling were immersed in 10 mM Tris (pH 7.0), 1 mM EDTA. Swabs were either processed immediately or stored until processing at −80° C. Swabs for DNA purification were processed using the MoBio PowerSoil® Kit, essentially as described by the manufacturer. Swabs for RNA purification were processed using the MoBio PowerMicrobiome™ RNA Isolation Kit as described by the manufacturer. Total RNA was depleted of human and microbial rRNA using the Epicentre/Illumina Ribo-Zero™ Magnetic Gold Kit as described by the manufacturer. DNA and RNA samples were stored at −80° C.

16S rRNA Taxonomic Surveys of the Vaginal Microbiome

Each DNA sample was amplified with barcoded primers validated for vaginal taxa as previously reported⁴⁸. Samples were multiplexed (384 samples/run) and sequenced using 2×300 b PE technology on an Illumina MiSeq® sequencer to generate a depth of coverage of at least 50,000 reads per sample. The raw sequence data was demultiplexed into sample paired-end fastq files based on unique barcode sequences using custom python script. The preprocessing of sequences was performed using the MeFiT⁵⁹ pipeline, with amplicons (on average ˜540 bp long) generated by merging the overlapping tails of paired-end sequences followed by quality filtering using a meep (maximum expected error rate) cutoff of 1.0. Non-overlapping high-quality reads were screened for chimeric sequences with UCHIME⁶⁰ against our custom database of vaginally relavant taxa. Each processed 16S rRNA gene sequence was taxonomically classified to the species-level by using STIRRUPS⁴⁸ by aligning against a custom reference database using USEARCH⁶¹. Reference sequences for Prevotella cluster 2 include Prevotella buccalis, Prevotella timonensis, Prevotella OTU46 and Prevotella OTU47. Only samples with at least 1,000 reads that met filtering criteria were analyzed.

Custom 16S rRNA V1-V3 Reference Database for Vaginal/Rectal Comparison

Full-length 16S rRNA gene sequences were collected from various sources—(i) the All-species Living Tree Project (Silva: LTPs123_SSU)⁶², (ii) STIRRUPS⁴⁸ database, and (iii) Human Oral Microbiome Database (HOMD)⁶³. Since the partial 16S rRNA V1-V3 region is not distinct enough to get species-level stratification for certain bacterial genera, we extracted the V1-V3 region from every reference sequence using V-Xtractor⁶⁴, a tool that identifies the hypervariable regions using Hidden Markov Models (HMMs). These partial sequences were then clustered into representative sequence set at 99% identity using UCLUST⁶¹. The representative sequences were annotated to the least common ancestor (LCA) taxonomic-level, in cases where the cluster comprised sequences from different bacterial species. The dataset was then supplemented by partial V1-V3 sequences from the STIRRUPs database, especially ones for which full length 16S rRNA sequence is not available. This resulted in a set of 9,299 representative 16S rRNA V1-V3 partial sequences. First sample visits (FIG. 1) were used to compare the proportional abundance of taxa in vaginal and rectal samples. Samples for 11 subjects did not have 16S rRNA profiles that passed the QC, thus 124 samples were used for this comparative analysis.

### Whole Metagenomic/Metatranscriptomic Sequencing

DNA libraries were prepared using KAPA Biosystems HyperPlus® Library Kit and sequenced on an Illumina HiSeq® 4000 (2×150 b PE). We multiplexed 24 samples per lane and obtain ˜1-2×10⁷ 150 nt reads per sample. The rRNA depleted messenger RNA was prepared for sequencing by constructing cDNA libraries using the KAPA Biosystems KAPA RNA HyperPrep® Kit. Indexed cDNA libraries were pooled in equimolar amounts and sequenced on the Illumina HiSeq® 4000 instrument running 4 multiplexed samples per lane with an average yield of ˜100 Gb/lane, sufficient to provide >100× coverage of the expression profiles of the most abundant 15-20 taxa in a sample.

### Whole Shotgun Metagenomic/Metatranscriptomic Data Pre-Processing

Raw sequence data was demultiplexed into sample-specific fastq files using bcl2fastq conversion software from Illumina. Adapter residues were trimmed from both 5′ and 3′ end of the reads using Adapter Removal tool v2.1.3⁶⁵. The sequences were trimmed for quality using meeptools⁶⁶, retaining reads with minimum read length of 70b and meep (maximum expected error) quality score less than 1. Human reads were identified and removed from each sample by aligning the reads to hg19 build of the human genome using the BWA aligner⁶⁷.

### Functional Analysis of Metagenomic and Metatranscriptomic Sequence Reads

Assignment of metagenomic and metatranscriptomic sequence reads to known genes/pathways was performed using ASGARD⁶⁸, HUMAnN2⁶⁹ and ShortBRED⁷⁰. These reads are also compared to appropriate databases (KEGG, GO, COG, etc.) using BLAST⁷¹ or other alignment tools to characterize functional data about these samples.

BVAB1 Genome Assembly from Metagenomic Reads

Starting with high quality trimmed metagenomic sequence reads from one sample with a high abundance of BVAB1, human reads were removed by alignment to the human hg19 reference sequence using BWA⁶⁷ alignment software. The remaining reads were processed as described below. The reads were digitally normalized with BBMap (sourceforge.net/projects/bbmap/) with a target coverage of >40× coverage to remove reads from highly repetitive elements of the genomes that may hamper the de novo assembly process and to ensure that reads originating from PCR duplication are excluded prior to assembly. Reads were assembled with SPAdes ver 3.8.0^{72,73} using the “-meta” option to generate a consensus assembly scaffold. Prior to clustering the scaffolds generated by SPAdes version 3.8.0, the human depleted reads were aligned back to the scaffolds using Bowtie2⁷⁴ with the ‘-very-sensitive’ option for global alignment. The resulting bam files were converted into “scaffold-to-average coverage” maps using a custom Python script. These contigs were clustered into individual genomes using MyCC⁷⁵ with tetramer frequencies coupled with the average coverage. Assemblies were identified by alignment with cpn60 or grpE genes. Note that ribosomal genes are too similar to segregate into different clusters. Reads were mapped back to individual MyCC clusters and then submitted to a new assembly using Newbler Assembler v2.8. Where necessary, gaps were closed by sequencing of PCR amplicons using primers directed to contig ends. Prokka⁷⁶ and ASGARD⁶⁸ were run on each assembly to find its gene repertoire and annotate it.

TM7-H1 Genome Assembly from Metagenomic Reads Using PacBio Single Molecule Long Read Technology.

DNA from a sample with high proportional abundance of TM7 was sent to Jonas Korlach (at Pacific Biosciences) for PacBio sequencing using the TdT protocol, which is suitable for sequencing of low-input samples. An HGAP metagenome assembly was performed using a white list to exclude reads mapped to human yielded three TM7 contigs. PCR amplication was performed across contigs gaps.

Identification of Virulence and Defense Genes of BVAB1, TM7-H1, Prevotella timonensis and Sneathia amnii

The genome sequences were annotated using an in-house pipeline that utilizes existing tools (e.g., Prokka⁷⁶, ASGARD⁶⁸, tRNAScan⁷⁷, RNAmmer⁷⁸). We also submitted sequences to the Rapid Annotation using Subsystem Technology (RAST) server⁷⁹ for genome annotation, which classified annotated genes into broad functional subsystems. We manually curated a virulence and defense functional supersystem category which included categories of genes that may cause pathogenic outcome, persistence, and/or help defend organisms from host and other bacterial species attack mechanisms. RAST identified functional subsystem categories in this curated supersystem included: virulence disease and defense, iron acquisition and metabolism, motility and chemotaxis, dormancy and sporulation, stress response, and sulfur metabolism. The list of genes in the supersystem were then used to compare across genomes and further used in comparisons of transcription levels of virulence genes in vaginal microbial profiles that included these taxa.

Transcript Abundances of BVAB1, TM7-H1, Prevotella timonensis, and Sneathia Amnii

Transcriptomic profiling of BVAB1, TM7-H1 Prevotella timonensis, Sneathia amnii was performed using PanPhlAn⁸⁰, a pangenome-based tool. First, we generated species-specific pangenomes; metagenomic reads were mapped against the corresponding pangenome using the software to obtain a gene family presence/absence matrix specific to a strain in a sample based on the coverage of all genes in a gene family cluster. For the samples for which we had matching metagenomics and metatranscriptomics data of the species at a minimum of 2× coverage, strain-specific transcriptional rates based on the gene family profiles from metagenomic sample data were generated using PanPhlAn. Virulence gene families were annotated by mapping to the virulence and defense functional supersystem category as called by RAST genome annotation of reference genomes.

### Metabolic Modeling

Draft constraint-based metabolic models for TM7, BVAB1, and Lactobacillus crispatus were generated using functional annotation information using EC numbers to describe function and KEGG IDs for nomenclature.

### Cytokine Profiling

The Bio-Plex Pro Human Cytokine 27-plex Assay panel (M50-0KCAF0Y, Bio-Rad, Hercules, Calif.) was used to measure cytokine concentrations according to manufacturer's protocol. Briefly the frozen vaginal swab samples were thawed on ice and centrifuged at 10,000×g for 10 min at 4° C. and diluted 4 fold in 100 mM Tris buffer, pH 7.5. The assay was carried out on a black 96-well plate (10021013, Bio-Rad, Hercules, Calif.), and 50 μl of cytokine standard, inter assay QC control (described below) and sample was added in duplicate to appropriate wells. The Bio-Plex MAGPIX® Multiplex Reader was used for data acquisition with default settings. Bio-Plex Manager 6.0 software was used for data analysis using five parameter logistic (5-PL) non-linear regression model upon optimization for all analytes within 70-130% recovery range.

The inter assay QC control was prepared from LPS stimulated cell culture medium. Briefly VK2/E6E7 (ATCC CRL-2616, ATCC Manassas, Va.) cells were initially grown in T75 flasks in DMEM/F12 supplemented with 10% FBS (11320-033, 26140079, ThermoFisher, Waltham, Mass.) at 37° C., 5% CO2 to confluency. These cells were trypsinized and reseeded at concentration of 3×10⁵ cells/ml per well on a 24-well plate (82050-892, VWR Radnor, Pa.). After 24 hrs, the medium was replaced with the fresh medium containing 100 ng/ml LPS (L2630-10MG, Sigma, St. Louis, Mo.). Twenty four hours post LPS treatment the cell culture medium was harvested, pooled and centrifuged at 3,000 rpm×10 min at 4° C. The resultant soluble fraction was aliquoted and stored at −80° C. for use as assay QC.

Out-of-range cytokine concentration values were imputed with the upper or lower limit of detection for the specific cytokine where necessary. Nine cytokines—IL-1b, Eotaxin, IL-8, TNF-α, IL-17A, MIP-1b, IL-6, IP-10, RANTES—had fewer than 30% out-of-range values and were selected for analysis.

### Statistical Analyses

### Community State Types (CSTs)/Vagitypes

Vaginal 16S rRNA profiles were assigned to CST/vagitype based on the taxon with the largest proportion of reads. Samples where the largest proportion was less than 30% were not assigned a CST/vagitpe. This “predominant taxon” rule has been shown to exhibit over 90% agreement with clustering-based methods across a variety of vaginal microbiome datasets⁸¹, yet is not population or dataset dependent and is therefore more conducive to use in a clinical setting. Differences in the numbers of L. crispatus CSTs among the PTB and TB cohorts was tested with a Fisher's exact test.

### Markov Chain Analysis

The R package msm was used to fit a continuous-time Markov chain model for CST transitions. The model takes as input the subject, CST/vagitype, and gestational age in days for each sample. The states were L. crispatus, L. iners, BVAB1, G. vaginalis, and “Other”. The pregnancy outcome (PTB, TB) was included as a co-variate. Because of low numbers of observed transitions between certain states, only point estimates (and not confidence intervals) were derived.

### Filtering Out Low-Abundant Taxa

As the first step in analyzing each dataset of vaginal 16S rRNA profiles, we analyzed the abundance of each taxa present in the profiles, and removed from further consideration low abundant species. We used two abundance criteria: we retained taxa that either a) 5% of the profiles exhibited an abundance of at least 1%, orb) at least 15% of profiles exhibited an abundance of at least 0.1%. Taxa that failed to meet both a) and b) were removed.

### Univariate Analysis to Identify Taxa Significantly Different in Abundance in Preterm and Term Birth Cohorts

We analyzed vaginal 16S rRNA profiles from 135 participants, 45 who delivered preterm and 90 who delivered term. The microbiome profile of the earliest sample from each of these women was used in this analysis. In this data set, 26 taxa remained after filtering out of the low-abundance taxa. For each of these 26 most abundant taxa, we performed a Mann-Whitney U test to identify significant differences in presence and abundance in preterm and term birth cohorts. For this analysis, abundance values below 0.00001 were rounded to zero. Taxa abundance was considered significantly different between cohorts if the q-value was less than a false discovery rate (FDR) of 5% after correction via the Benjamini-Hochberg procedure⁸². For each taxon, we also calculated median and 75-percentile in the preterm and term birth cohorts.

### Longitudinal Models

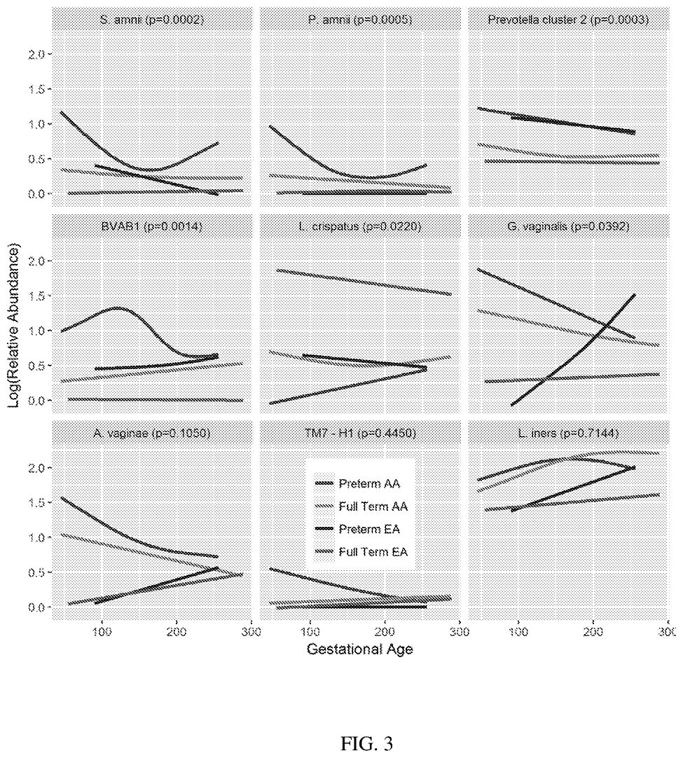

A generalized additive mixed model (GAMM)⁸³ incorporating BMI, ethnicity (African, European), pregnancy outcome (pre-term, full-term), a smoother for gestational age, and a random subject effect was used to longitudinally model log transformed relative abundances of vaginally relevant taxa (FIG. 3). Effect contributions were determined using ANOVA tests. The degree of smoothness for gestational age was estimated by restricted maximum likelihood⁸⁴. Models were fit using the gamm4: Generalized additive mixed models using mgcv and lme4. R package package in R released by Wood and Scheipl in 2017.

### Canonical Correlation Analysis of Cytokines and Vaginal Microbiome Profiles

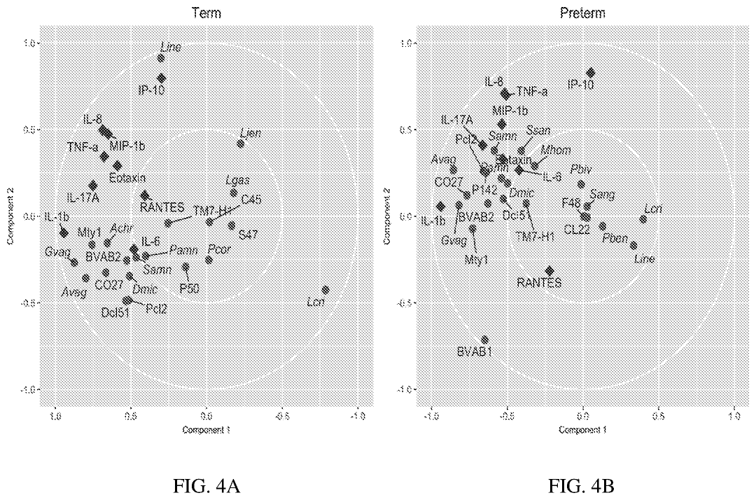

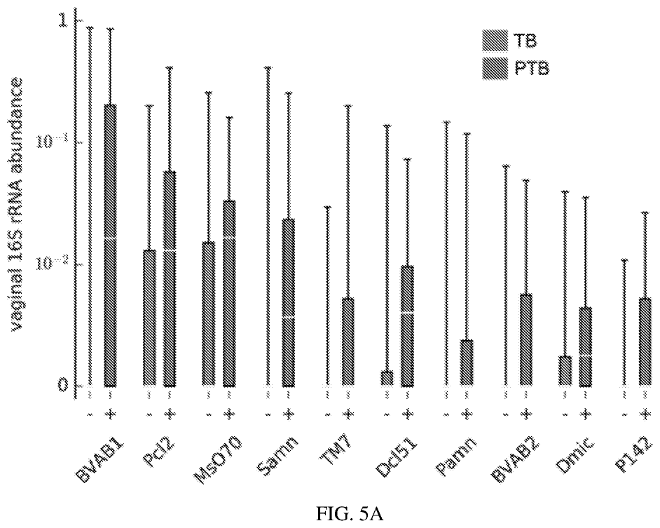

An integrative analysis of both log-transformed 16S rRNA survey data and log-transformed cytokine data was performed using sparse canonical correlation analysis^{85,86} (sCCA, FIG. 4). Classical canonical correlation analysis⁸⁷ explores the correlation between two sets of quantitative variables measured on the same subjects. sCCA introduces an 11 penalization term to handle the case of more variables than observations. Nine cytokines, with fewer than 30% out-of-range values, were selected for analysis (IL-1b, Eotaxin, IL-8, TNF-α, IL-17A, MIP-1b, IL-6, IP-10, RANTES). Out-of-range cytokine concentration values were then imputed with the upper or lower limit of detection for the specific cytokine where appropriate. For each subject, the observation corresponding to the earliest gestational age per trimester was used for analysis. We perform sCCA separately for full term and pre-term subjects using the sgcca function in the R package mixOmics⁸⁸.

The results of sCCA are displayed in a correlation circle plot⁸⁷. The coordinates of the plotted points (variables) are the correlations between the variables and their canonical variates. Variables that are strongly positively correlated are projected close to each other on the plot while variables that are negatively correlated are plotted opposite each other. The greater the distance from the origin, the stronger the relationship among variables⁸⁷. The correlation circle plots are constructed using the plotVar function in the R package mixOmics.

### Taxa Co-Occurrence

Bacterial taxa were determined to be present if they comprised greater than or equal to 0.1% of the total vaginal microbiome profile. We utilized the statistical tool REBACCA⁸⁹ to mitigate the effects of relative constraint. REBACCA was run using 50 bootstraps and a visualization of bacterial correlations was generated using Gephi⁹⁰. Correlations with greater than 0.30 or less than −0.3 are shown with negative correlations. Edge weights are representative of the strength of correlation between taxa and the four major predictive taxa.

### Predictive Modeling of Preterm Birth Using Early-Pregnancy Microbiome Profiles

We constructed a linear predictive model of preterm birth as follows. From the full cohort, we selected subjects who had at least one vaginal 16S rRNA sample early in the pregnancy, in the day 42-day 167 (inclusive) gestational age range. A total of 31 PTB and 59 TB subjects had at least one sample in this time window; if multiple samples were present in that window, we used the earliest sample.

We first filtered out low-abundant species in this dataset: 25 passed the selection criteria. For these taxa, the abundance data was soft-thresholded with 0.001 threshold, to reduce the impact of statistical noise resulting from low-abundance values, by subtracting 0.001 from the abundance and setting all resulting negative values to 0, and log-transformed through a transform log 10((abundance+0.001)/0.001), where dividing by 0.001 shifts the logarithm values for abundances in the zero (0.0) to one (1.0) range from negative to non-negative values. Ten taxa were significantly different between the PTB and TB cohorts.

The model construction uses a two-step procedure. First, we applied a Mann-Whitney U test to all species that survived the abundance-based filtering criteria, retaining species with a two-sided p-value of 0.05 or less. Based on these species, the predictive model was trained using logistic regression with L₁ regularization⁹¹, to reduce the impact of collinearity between species and the resulting sign-reversals and false detections. Regularized logistic regression finds a vector of taxa weights w that minimizes: Σᵢln(1+exp(−yᵢwxᵢ))+C∥w∥₁ over the training set of samples (xᵢ, yᵢ). The constant C was selected based only on samples from the training set, using grid search and nested cross-validation.

The statistical significance of the model in the form of a p-value was estimated using a permutation test, consisting of training 10,000 models on data with the class variable randomly permuted prior to processing, and comparing the distribution of the 10,000 auROC values with the auROC of the original model trained using unperturbed class variable. Performance of the model on previously unseen samples was assessed using 200 independent runs of 5-fold stratified cross-validation, for a total of 1000 training set—test set pairs. We assessed test set sensitivity, specificity, and area under ROC curve (auROC), as implemented in the python scikit-learn package⁹². For each of these metrics, we calculated the average and standard deviation over the 1000 models.

### Nucleotide Sequence Accession Numbers

The sequences of vaginal strains of Sneathia amnii SN35 (accession: NZ_CP011280) and Prevotella timonensis CRIS-5C-B1 (accession: NZ_ADEF01000048) as well as recent submission of BVAB1 S1 (PQV000000000) and TM7-H1 E1 (CP026537) are available in the Genbank database.

### Results

### Vaginal Microbiome Profiles Show Preterm Birth-Associated Trends

In our longitudinal iHMP study, the Multi-Omic Microbiome Study—Pregnancy Initiative (MOMS-PI), we enrolled 1,594 pregnant women from clinics associated with the Research Alliance for Microbiome Science Registry based at Virginia Commonwealth University in Virginia and the Global Alliance to Prevent Prematurity and Stillbirth in Washington. From this cohort, we analyzed 45 single gestation pregnancies that met criteria for experiencing spontaneous PTB and 90 single gestation pregnancies that extended through term (≥39 weeks). The TB controls were matched for age, race, and annual household income for analysis. Participants were recruited during prenatal visits and samples were longitudinally collected either by clinicians during a pelvic exam or by self-sampling. On average, the earliest samples were collected at 18 weeks gestation, and the mean number of sampling visits per participant was seven.

The cohort is predominantly comprised of women of African ancestry (78%), with a median annual income less than S20,000 and an average age of 26 years (Table 1).

TABLE 1
Description of cohort studied in this project.

| | |
|---|---|
| Preterm | Term |
| delivery <37 | delivery ≥39 |
| wks | wks |
| (N = 45) | (N = 90) |

| | | | | |
|---|---|---|---|---|
| Mean age in years* | 26 | (5.68) | 25.9 | (5.43) |
| Ancestry/Ethnicity | | | | |
| African | 35 | (77.8%) | 71 | (78.9%) |
| European | 6 | (13.3%) | 13 | (14.4%) |
| Hispanic | 3 | (6.7%) | 5 | (5.6%) |
| Native American | 1 | (2.2%) | 1 | (1.1%) |
| Household Income* | | | | |
| <20,000 | 29 | (72.5%) | 66 | (77.7%) |
| 20,000-59,999 | 9 | (22.5%) | 15 | (17.6%) |
| 60,000+ | 2 | (5.0%) | 4 | (4.7%) |
| Vaginal Delivery | 38 | (84.4%) | 74 | (82.2%) |
| Previous Preterm | 14 | (31.1%) | 10 | (11.1%) |
| PPROM | 26 | (57.8%) | 0 | (0% |

*Standard deviation listed in in parentheses

Microbiome profiles of the first vaginal samples collected at study enrollment (FIG. 1) were generated by 16S rRNA. Most profiles were classified into one of several common community state types, or vagitypes, dominated by Lactobacillus crispatus, Lactobacillus iners, other lactobacilli (e.g., Lactobacillus delbrueckii, Lactobacillus gasseri), A. vaginae, Lachnospiraceae BVAB1, G. vaginalis, and a complex vagitype with no predominant taxon (FIG. 1). A significantly smaller proportion of women who would later experience PTB had a vaginal microbiome characterized by prevalence of L. crispatus compared with women who would later deliver at term (p=0.014, FIG. 1b). This result parallels earlier observations²⁷ that associate Lactobacillus species with a more protective state of the vaginal microbiome. A Markov chain analysis to assess vagitype changes throughout pregnancy reveals that women who would later deliver prematurely were more likely to transition to a vagitype characterized by BVAB1 and less likely to transition to a vagitype characterized by L. crispatus or L. iners.

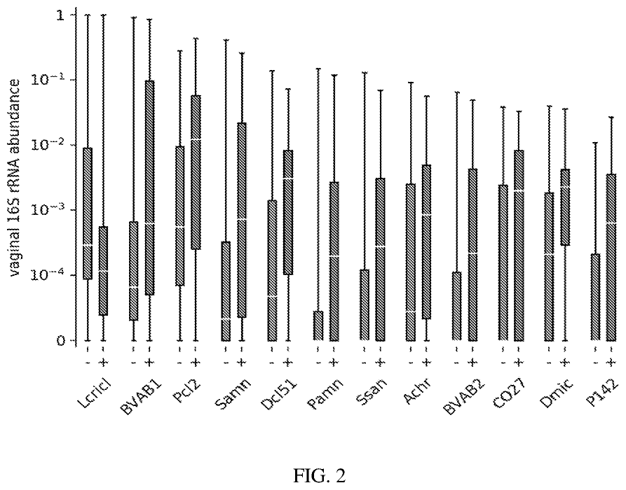

The analysis described above identifies differences in predominant vagitypes associated with women who will experience PTB. To identify specific taxa associated with PTB, we compared the abundance of 26 abundant taxa found in these microbiome profiles using the earliest samples taken from each of the 45 women who experienced PTB and the 90 TB controls. We found that overall diversity was increased in PTB and twelve taxa showed a significant difference between the PTB and the TB groups. L. crispatus was significantly reduced in PTB samples, and several other taxa, including BVAB1, Prevotella cluster 2 and S. amnii, were more abundant in PTB samples (q<0.05, FIG. 2). Prevotella cluster 2 is comprised of several closely related Prevotella species⁴⁸, and we found that most of these sequences mapped to the reference sequence for Prevotella timonensis. An analysis using only enrollment samples that were taken in the first 24 weeks of pregnancy identified two additional taxa, Megasphaera type 1 and TM7-H1 (i.e., BVAB-TM7-H1), two bacterial previously associated with adverse conditions of vaginal health²³, as significantly increased in PTB. These findings extend those of a previous study that found BVAB1 and Sneathia species carriage in early and mid-pregnancy to be associated with spontaneous preterm birth³⁶. To our knowledge, this is the first report of an association of TM7-H1 with PTB.

We and others^{29,49,50} have shown that predominance of Lactobacillus species tends to be more stable in pregnancy, which is possibly an evolutionary adaptation by which physiological changes that occur during pregnancy favor an environment that reduces the abundance of potentially harmful bacterial species in the female reproductive tract. Considering that an adverse pregnancy outcome may be caused by a breakthrough of pathogenic microbes, this trend suggests that microbiome composition early in pregnancy may be most useful in prediction of adverse outcomes. We examined the longitudinal trends of several of the taxa significantly correlated, both positively and negatively, with PTB. FIG. 3 shows the results of a generalized additive mixed effect model (GAMM) incorporating BMI, ethnicity (i.e., African or European ancestry), pregnancy outcome (i.e, PTB, TB), a smoother for gestational age, which captures the major trends while leaving out noise and fine-scale structures in the data, and a random subject effect to longitudinally model log-transformed relative abundances of vaginally relevant taxa. As we³² and others⁵¹ have noted, the vaginal microbiome profiles of women of African and European ancestry differ significantly, and we therefore stratified the analysis by ancestry. In this analysis, BVAB1, G. vaginalis, L. crispatus, P. amnii, Prevotella cluster 2, and S. amnii were significantly different in women who deliver preterm or term. Moreover, based on GAMM fit, women of African ancestry who deliver preterm experience significant decreases, over the term of pregnancy, in prevalence of A. vaginae (p=0.005), BVAB1 (p=0.0001), G. vaginalis (p=0.0001), P. amnii (p=0.0003), S. amnii (p=0.017) and TM7-H1 (p=0.001). Women of African ancestry who delivered full term exhibited fewer changes in the modeled taxa throughout pregnancy, although decreases in prevalence of A. vaginae (p=0.0001) and G. vaginalis (p=0.0001), and an increase in L. iners (p=0.015) were observed. Women of European ancestry exhibited microbiome profiles with greater apparent stability during pregnancy, although an increase in prevalence of G. vaginalis (p=0.002) was noted for women who delivered preterm. Women of African ancestry have a significantly increased risk of PTB compared to women of European ancestry. Thus, we may expect that with a case-control study design, carriage of taxa with intermediate risk, such as the phylotypes of G. vaginalis, may be associated with PTB in a lower-risk cohort, but TB in a higher-risk cohort. We previously reported³² that carriage of L. crispatus, which is negatively associated with PTB, is more prevalent in women of European ancestry, and BVAB1, which is positively associated with PTB, is more common in women of African ancestry. Thus, our findings are consistent with a proposed framework in which there exists a comprehensive spectrum of vaginal microbiome states linked to risk for preterm birth. Note that relative abundance of bacterial taxa associated with PTB is generally quite low in the early stages of pregnancy in women of African ancestry who experience TB, as well most women of European ancestry, and significant decreases may therefore be difficult to detect (FIG. 3).

Vaginal samples from each of the participants were subjected to metagenomic sequencing and a subset of those samples that were collected between 14 and 27 weeks gestation were also subjected to metatranscriptomic analysis as outlined in the Methods. A pathway analysis identified a relatively conserved functional and metabolic potential of the microbial communities, independent of vagitype, across the samples. However, the proportional transcriptional activity devoted to each pathway varied. For example, L. crispatus-dominated samples had significantly higher proportional transcriptional activity of UDP-N-acetyl-D-glucosamine biosynthesis, which is involved in production of peptidoglycan. Peptidoglycan is one of the best described microbe-associated molecular patterns (MAMPs) involved in the modulaton of host cytokine production via Toll-like receptor signaling. Conversely, the proportional transcriptional activity of genes classified to the pyruvate fermentation to acetate and lactate II and the non-oxidative branch of the pentose phosphate pathway was lower in L. crispatus-dominated samples. This finding is consistent with previous reports of reduced levels of lactic acid and increased concentrations of short-chain fatty acids (SCFA) (e.g., acetate, propionate, butyrate, and succinate) in vaginal samples of women with bacterial vaginosis. Intriguingly, SCFAs have been suggested to reduce anti-microbial activity and promote proinflammatory cytokines in the vaginal environment⁵².

Bacterial Taxa Associated with PTB Encode and Express Potential Virulence Factors

Metagenomic sequence data were assembled to characterize the genomes of BVAB1 and TM7-H1, respectively. To our knowledge, these taxa have not been previously cultivated or characterized beyond their 16S rRNA sequences. We examined these genomes along with the available genomes of S. amnii SN35 (NZ_CP011280), which we previously reported⁵³, and P. timonensis CRIS-5C-B1 (NZ_ADEF01000048). The genome sizes were ˜0.72 Mb for TM7-H1 (CP026537), ˜1.34 Mb for S. amnii (CP011280.1), ˜1.45 Mb for BVAB1 (PQV000000000), and ˜2.8 Mb for P. timonensis (NBAX01000001). BVAB1, P. timonensis, and S. amnii are classified to the Lachnospiraceae, Leptotrichiaceae, and Prevotellaceae families, respectively. TM7-H1 falls into the Candidatus Saccharibacteria phylum and exhibits ˜66% nucleotide identity to the recently described oral TM7x (NZ_CP007496). TM7-H1 encodes a putative alpha-amylase and is predicted to be able to utilize glycogen as a carbon source. Like TM7x⁵⁴, TM7-H1 lacks de novo biosynthetic capabilities for essential amino acids and likely depends on other organisms in the vaginal environment for survival. TM7x has been characterized as an obligate, parasitic epibiont; it is unknown whether TM7-H1 similarly lives on the surface of another bacterial species in the vaginal environment. Using these reference genomes, we mapped taxon-specific transcripts from metatranscriptomic data from the vaginal samples of PTB and TB controls and observed that, although there was some variation in the activities of several metabolic and signaling pathways, the overall transcriptional profiles of each of these taxa were largely conserved across both PTB cases and TB controls.

We looked at broad classes of genes with possible roles in virulence and defense. Genes annotated with putative roles in antibiotic resistance, resistance to toxic compounds and oxidative stress were identified in all four genomes. Other putative defenses were variable; for example, 57 genes predicted to be involved in motility were identified in BVAB1, many of which were transcribed at high levels in vaginal samples. There was no obvious enhancement of expression levels of the virulence genes in women who would later experience PTB, a largely expected result since expression levels are likely controlled by general conditions of the vaginal environment. We further performed a metabolic reconstruction and modeling of TM7-H1 and BVAB1. We identified 243 metabolic reactions in TM7-H1 and and 421 metabolic reactions in BVAB1. Both of the organisms are predicted to have the ability to produce pyruvate, acetate, L-lactate and propionate; BVAB1 encodes additional pathways for production of acetaldehyde, D-lactate formate, and acetyl-CoA. Neither is predicted to take part in the TCA cycle, and TM7-H1 completely lacks genes related to butyrate metabolism. As described above, production of SCFAs has been linked to a proinflammatory state⁵², with possible implications for disease. As such, these metabolic models lay the foundations for understanding possible mechanisms by which these bacteria impact pregnancy. Taken together, identification of these virulence genes and factors in these four bacterial strains is consistent with our finding that they are associated with PTB. More importantly, characterization of the genomes of these strains and identification of likely determinants of pathogenicity represents an important step toward understanding the mechanisms by which components of the vaginal microbiome mediate or cause PTB.

### Host Cytokine Expression in PTB

To examine the roles of cytokines in the progression of pregnancy to PTB, we measured cytokine levels in vaginal swab samples. Using the cytokine data from nine (IL-1b, IL-6, IL-8, Eotaxin, TNF-α, IL-17A, MIP-1b, IP-10, RANTES) of the 27 examined, and data from the 16S rRNA taxonomic surveys, both log transformed, we performed an integrative sparse canonical correlation analysis to assess the correlation between bacterial taxa and cytokine levels (FIG. 4). For each participant, the observation corresponding to the earliest gestational age per trimester was used in the analysis. In women who delivered at term (FIG. 4a), we observed a negative correlation between L. crispatus and the taxa associated with dysbiosis and PTB (e.g., G. vaginalis, Prevotella cluster 2, S. amnii, and to a lesser extent TM7-H1), as well as with the analyzed cytokines. The analyzed cytokines, with the exception of IP-10 are largely proinflammatory in function, were positively correlated both with each other and with taxa associated with dysbiosis and PTB. In contrast, we observed that IP-10 was positively associated with L. iners, an association that has been reported by others⁵⁵, and negatively associated with both L. crispatus and taxa associated with dysbiosis.

There were notable differences in the taxa-cytokine correlations in women who went on to experience PTB. The proinflammatory cytokines (e.g., IL-1b) and dysbiotic taxa (e.g. A. vaginae, G. vaginalis, and Megasphaera type 1) tend to form a tighter cluster. In contrast, IP-10 did not cluster with L. iners in the PTB cohort and BVAB1, which was not selected as a feature by the analysis in the TB group, was negatively correlated with IP-10 in PTB samples. These observations generally support the concept that bacterial taxa in women who experience PTB (e.g., Prevotella cluster 2, S. amnii and TM7-H1, among others) are positively correlated with proinflammatory cytokines, and negatively correlated with taxa (e.g., L. crispatus) that are negatively correlated with PTB.

### Predictive Model for PTB

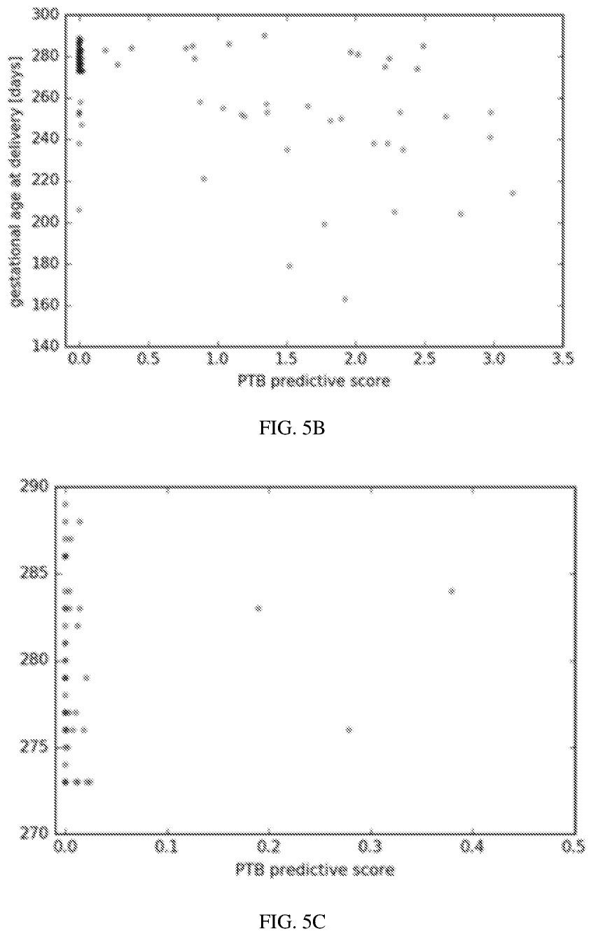

Early prediction of risk for PTB is critical for the development of new strategies for prevention and intervention. Using a set of 31 PTB and 59 TB subjects that had samples collected early (6-24 weeks gestational age) in pregnancy, we developed a predictive model for PTB. Model construction involved selecting taxa that are differentially represented in the PTB and TB cohorts as assessed using the Mann-Whitney U test (FIG. 5a), and assigning weights to these taxa using L₁-regularized logistic regression. The resulting model incorporates four taxa: S. amnii, BVAB1, Prevotella cluster 2, and TM7-H1, which are all positively correlated to PTB (FIG. 5a). The model is significant (p=0.0024) and has expected sensitivity of 76±17% (mean±SD), specificity of 74±13%, and an area under the ROC curve of 0.769±0.108 for samples not used during training. Thus, this modeling strategy represents a promising approach to utilization of microbiome data obtained early in pregnancy to identify pregnancies with higher risk of PTB for possible prophylaxis.

### Conclusions

Our comparison of the vagitypes in 45 women who experienced PTB to 90 women who experience TB revealed a significant difference in the proportion of samples characterized by predominance of L. crispatus. Consistent with this observation, we identified 12 taxa that were significantly associated with PTB in this cohort. L. crispatus, which is thought to play a generally protective role in the female reproductive tract, was negatively associated with PTB; the other 11 taxa, many of which have been implicated in vaginal dysbiosis and bacterial vaginosis, were positively correlated with PTB. BVAB1, Prevotella cluster 2, Sneathia amnii, and TM7-H1, were consistently identified as relevant to PTB in multiple analyses. A network analysis of these four taxa (FIG. 5b) show them to be positively correlated with taxa associated with vaginal dysbiosis. At the selected threshold, Prevotella cluster 2 was the only taxon that had negative correlations with Lactobacillus species, which is intruiging given that Prevotella species have been reported to be associated with PTB in both low-risk and high-risk cohorts of women of European and African ancestry³⁹. We further found that Prevotella species, including P. timonensis, showed higher relative abundance in rectal samples compared to vaginal samples, consistent with the hypothesis that a recto-vaginal pathway may play a role for some key taxa that are associated with preterm birth. These relationships indicate that neither the vagitype nor a small number of indicator bacteria are sufficient to fully characterize PTB risk, but rather complex interactions between bacterial communities and the host are important.

In women of African ancestry, taxa significantly associated with PTB were more highly correlated early in pregnancy. This finding is consistent with previous observations that pregnancy tends to be associated with reduced carriage of bacterial vaginosis-associated organisms. Our longitudinal modeling supported this concept in that taxa associated with PTB tended to decrease in abundance in the vaginal environment throughout pregnancy. We observed that women of African ancestry who experienced PTB often exhibited an initial decrease in prevalence of these taxa followed by a reappearance in the third trimester. This finding is consistent with a proposed model in which carriage of one or more of taxa associated with PTB in early pregnancy confers an increased risk for a subsequent breakthrough event that leads to infection and inflammatory processes that induce premature delivery.

Because the composition of the vaginal microbiome in early pregnancy may be more relevant to PTB, we explored a predictive model for PTB using taxonomic data from early pregnancy vaginal samples. The model focused on the three taxa that consistently showed an association with PTB (i.e., BVAB1, Prevotella cluster 2, S. amnii) and TM7-H1, which was not prominent in our initial analyses, probably because it is generally of lower abundance and tends to peak in very early pregnancy. Inclusion of TM7-H1 provided a significant enhancement to the model, which has promise for the early prediction of PTB with mean sensitivity and specificity of ˜75%.

The data suggest that the four taxa used on our predictive model (i.e., BVAB1, Prevotella cluster 2, S. amnii and TM7-H1) have roles in causation of PTB. We previously characterized the genome of S. amnii, identifying several potential cytotoxins, and showed that cultured bacteria kill eukaryotic cells in vitro⁵³. Moreover, analysis of our metagenomic genome assemblies of BVAB1 and TM7 and an available genome of P. timonensis identified multiple potential virulence factors, antibiotic resistance genes, episomal elements, and genes encoding proteins associated with cellular invasion and intracellular existence. These factors can now be readily genetically manipulated and tested for activity in heterologous systems. Our analysis of cytokine expression also represents an attempt to incorporate causal and mechanistic insight into the relationship between the vaginal microbiome and risk of PTB. Labor is associated with proinflammatory cytokine expression, and premature labor can be induced by inflammatory responses. Our analysis is consistent with previous findings and shows that bacterial taxa generally associated with dysbiosis and bacterial vaginosis are highly correlated with expression of pro-inflammatory cytokines, which may play a role in induction of labor. We also observed that vaginal IP-10 levels were inversely correlated with BVAB1 in PTB, inversely correlated with L. crispatus in TB and positively correlated with L. iners in TB cohort, suggesting that there exist complex host-microbiome interactions in pregnancy. Previous studies¹² disclosed the relative contributions of maternal and fetal genetics to PTB, and the impact of bacterial vaginosis, implicating gene-environment interactions that modulate the maternal and fetal immunological and inflammatory cascades, resulting in early labor and delivery. In apparent contradiction to the conclusion that bacteria play a role in triggering PTB, treatment of bacterial vaginosis and vaginal dysbiosis with the antibiotics, metronidazole or clindamycin, failed to prevent PTB^{56,57}. This may not be surprising, since it is becoming apparent that multiple bacterial taxa of widely different phylogeny are positively associated with PTB and that they may interact with each other and the host in complex biofilm-formations. Thus, a single antibiotic may not target all relevant taxa and might in fact clear the way for pathogenic culprits by eliminating their healthier competitors. The genomes of these bacteria may harbor mechanistic clues such as genes associated with antibiotic resistance, defense, and genes encoding putative surface proteins that may modulate microbe-microbe and microbe-host interactions. Further, more in-depth analysis of metabolic models may yield insight into how these taxa rely on each other and compete for resources within the vaginal environment. The findings described herein provide new insights and methods which can be used to prospectively assess risk of PTB and create strategies for prophylaxis treatment.

### REFERENCES

1. WHO|Preterm birth. WHO Available at: http://www.who.int/mediacentre/factsheets/fs363/en/. (Accessed: 1st November 2017)
2. Blencowe, H. et al. Born Too Soon: The global epidemiology of 15 million preterm births. Reprod. Health 10, S2 (2013).
3. Marret, S. et al. Neonatal and 5-year Outcomes After Birth at 30-34 Weeks of Gestation. Obstet. Gynecol. 110, 72-80 (2007).
4. Wolke, D., Eryigit-Madzwamuse, S. & Gutbrod, T. Very preterm/very low birthweight infants' attachment: infant and maternal characteristics. Arch. Dis. Child.—Fetal Neonatal Ed. 99, F70-F75 (2014).
5. Verrips, G. et al. Long term follow-up of health-related quality of life in young adults born very preterm or with a very low birth weight. Health Qual. Life Outcomes 10, 49 (2012).
6. Wolke, D. et al. Self and Parent Perspectives on Health-Related Quality of Life of Adolescents Born Very Preterm. J. Pediatr. 163, 1020-1026.e2 (2013).
7. Simms, V. et al. Mathematics difficulties in extremely preterm children: evidence of a specific deficit in basic mathematics processing. Pediatr. Res. 73, 236-244 (2013).
8. Murray, C. J. L. et al. Disability-adjusted life years (DALYs) for 291 diseases and injuries in 21 regions, 1990-2010: a systematic analysis for the Global Burden of Disease Study 2010. The Lancet 380, 2197-2223 (2012).
9. Blencowe, H., Lawn, J. E., Vazquez, T., Fielder, A. & Gilbert, C. Preterm-associated visual impairment and estimates of retinopathy of prematurity at regional and global levels for 2010. Pediatr. Res. 74, 35-49 (2013).
10. Manuck, T. A. Racial and ethnic differences in preterm birth: A complex, multifactorial problem. Semin. Perinatol. (2017). doi:10.1053/j.semperi.2017.08.010
11. Behrman, R. E., Butler, A. S. & Outcomes, I. of M. (US) C. on U. P. B. and A. H. Societal Costs of Preterm Birth. (National Academies Press (US), 2007).
12. York, T. P., Eaves, L. J., Neale, M. C. & Strauss, J. F. The contribution of genetic and environmental factors to the duration of pregnancy. Am. J. Obstet. Gynecol. 210, 398-405 (2014).
13. Preterm Birth: Causes, Consequences, and Prevention. (National Academies Press (US), 2007).
14. Goldenberg, R. L., Culhane, J. F., Jams, J. D. & Romero, R. Epidemiology and causes of preterm birth. Lancet Lond. Engl. 371, 75-84 (2008).
15. Donders, G. G. et al. Predictive value for preterm birth of abnormal vaginal flora, bacterial vaginosis and aerobic vaginitis during the first trimester of pregnancy. BJOG Int. J. Obstet. Gynaecol. 116, 1315-1324 (2009).
16. Martius, J. et al. Relationships of vaginal Lactobacillus species, cervical Chlamydia trachomatis, and bacterial vaginosis to preterm birth. Obstet. Gynecol. 71, 89-95 (1988).
17. What are the risk factors for preterm labor and birth? Available at: https://www.nichd.nih.gov/health/topics/preterm/conditioninfo/Pages/who_risk.aspx. (Accessed: 1st November 2017)
18. Romero, R., Dey, S. K. & Fisher, S. J. Preterm Labor: One Syndrome, Many Causes. Science 345, 760-765 (2014).
19. Cobb, C. M. et al. The oral microbiome and adverse pregnancy outcomes. Int. J. Womens Health 9, 551-559 (2017).
20. Pretorius, C., Jagatt, A. & Lamont, R. F. The relationship between periodontal disease, bacterial vaginosis, and preterm birth. J. Perinat. Med. 35, 93-99 (2007).
21. Parihar, A. S. et al. Periodontal Disease: A Possible Risk-Factor for Adverse Pregnancy Outcome. J. Int. Oral Health JIOH 7, 137-142 (2015).
22. Structure, function and diversity of the healthy human microbiome. Nature 486, 207-214 (2012).
23. Fredricks, D. N., Fiedler, T. L., Thomas, K. K., Oakley, B. B. & Marrazzo, J. M. Targeted PCR for Detection of Vaginal Bacteria Associated with Bacterial Vaginosis. J. Clin. Microbiol. 45, 3270-3276 (2007).
24. Sobel, J. D. Bacterial vaginosis. Annu. Rev. Med. 51, 349-356 (2000).
25. Bradshaw, C. S. & Sobel, J. D. Current Treatment of Bacterial Vaginosis—Limitations and Need for Innovation. J. Infect. Dis. 214, S14-S20 (2016).
26. Chavoustie, S. E. et al. Experts explore the state of bacterial vaginosis and the unmet needs facing women and providers. Int. J. Gynecol. Obstet. (2017). doi:10.1002/ijgo.12114
27. Ma, B., Forney, L. J. & Ravel, J. The vaginal microbiome: rethinking health and diseases. Annu. Rev. Microbiol. 66, 371-389 (2012).
28. Hickey, R. J., Zhou, X., Pierson, J. D., Ravel, J. & Forney, L. J. Understanding vaginal microbiome complexity from an ecological perspective. Transl. Res. J. Lab. Clin. Med. 160, 267-282 (2012).
29. MacIntyre, D. A. et al. The vaginal microbiome during pregnancy and the postpartum period in a European population. Sci. Rep. 5, 8988 (2015).
30. Ravel, J. et al. Vaginal microbiome of reproductive-age women. Proc. Natl. Acad. Sci. U.S.A 108 Suppl 1, 4680-4687 (2011).
31. Martin, D. H. & Marrazzo, J. M. The Vaginal Microbiome: Current Understanding and Future Directions. J. Infect. Dis. 214, S36-S41 (2016).
32. Fettweis, J. M. et al. Differences in vaginal microbiome in African American women versus women of European ancestry. Microbiol. Read. Engl. 160, 2272-2282 (2014).
33. Zhou, X. et al. Differences in the composition of vaginal microbial communities found in healthy Caucasian and black women. ISME J. 1, 121-133 (2007).
34. Hyman, R. W. et al. Diversity of the Vaginal Microbiome Correlates With Preterm Birth. Reprod. Sci. 21, 32-40 (2014).
35. Beamer, M. A. et al. Bacterial species colonizing the vagina of healthy women are not associated with race. Anaerobe 45, 40-43 (2017).
36. Nelson, D. B. et al. Early Pregnancy Changes in Bacterial Vaginosis-Associated Bacteria and Preterm Delivery. Paediatr. Perinat. Epidemiol. 28, 88-96 (2014).
37. Romero, R. et al. The vaginal microbiota of pregnant women who subsequently have spontaneous preterm labor and delivery and those with a normal delivery at term. Microbiome 2, 18 (2014).
38. DiGiulio, D. B. et al. Temporal and spatial variation of the human microbiota during pregnancy. Proc. Natl. Acad. Sci. U.S.A 112, 11060-11065 (2015).
39. Callahan, B. J. et al. Replication and refinement of a vaginal microbial signature of preterm birth in two racially distinct cohorts of US women. Proc. Natl. Acad. Sci. U.S.A 114, 9966-9971 (2017).
40. Brown, R. et al. Role of the vaginal microbiome in preterm prelabour rupture of the membranes: an observational study. The Lancet 387, S22 (2016).
41. Nelson, D. B., Shin, H., Wu, J. & Dominguez-Bello, M. G. The Gestational Vaginal Microbiome and Spontaneous Preterm Birth among Nulliparous African American Women. Am. J. Perinatol. 33, 887-893 (2016).
42. Stout, M. J. et al. Early pregnancy vaginal microbiome trends and preterm birth. Am. J. Obstet. Gynecol. 217, 356.e1-356.e18 (2017).
43. DiGiulio, D. B. Diversity of microbes in amniotic fluid. Semin. Fetal. Neonatal Med. 17, 2-11 (2012).
44. Goldenberg, R. L. et al. The Alabama Preterm Birth Study: Umbilical cord blood Ureaplasma urealyticum and Mycoplasma hominis cultures in very preterm newborn infants. Am. J. Obstet. Gynecol. 198, 43.e1-43.e5 (2008).
45. Han, Y. W., Shen, T., Chung, P., Buhimschi, I. A. & Buhimschi, C. S. Uncultivated bacteria as etiologic agents of intra-amniotic inflammation leading to preterm birth. J. Clin.

Microbiol. 47, 38-47 (2009).
46. Kindinger, L. M. et al. The interaction between vaginal microbiota, cervical length, and vaginal progesterone treatment for preterm birth risk. Microbiome 5, 6-6 (2017).
47. The Integrative Human Microbiome Project: Dynamic Analysis of Microbiome-Host Omics Profiles during Periods of Human Health and Disease. Cell Host Microbe 16, 276-289 (2014).
48. Fettweis, J. M. et al. Species-level classification of the vaginal microbiome. BMC Genomics 13 Suppl 8, S17 (2012).
49. Romero, R. et al. The composition and stability of the vaginal microbiota of normal pregnant women is different from that of non-pregnant women. Microbiome 2, 4 (2014).
50. Walther-Antonio, M. R. S. et al. Pregnancy's stronghold on the vaginal microbiome. PloS One 9, e98514 (2014).
51. Borgdorff, H. et al. The association between ethnicity and vaginal microbiota composition in Amsterdam, the Netherlands. PloS One 12, e0181135 (2017).
52. Aldunate, M. et al. Antimicrobial and immune modulatory effects of lactic acid and short chain fatty acids produced by vaginal microbiota associated with eubiosis and bacterial vaginosis. Front. Physiol. 6, 164 (2015).
53. Harwich, M. D., Jr et al. Genomic sequence analysis and characterization of Sneathia amnii sp. nov. BMC Genomics 13 Suppl 8, S4 (2012).
54. He, X. et al. Cultivation of a human-associated TM7 phylotype reveals a reduced genome and epibiotic parasitic lifestyle. Proc. Natl. Acad. Sci. U.S.A 112, 244-249 (2015).
55. Jespers, V. et al. A longitudinal analysis of the vaginal microbiota and vaginal immune mediators in women from sub-Saharan Africa. Sci. Rep. 7, 11974 (2017).
56. Haahr, T. et al. Treatment of bacterial vaginosis in pregnancy in order to reduce the risk of spontaneous preterm delivery—a clinical recommendation. Acta Obstet. Gynecol. Scand. 95, 850-860 (2016).
57. Myrhaug, H. T., Brurberg, K. G., Kirkehei, I. & Reinar, L. M. Treatment of Pregnant Women with Asymptomatic Bacterial Vaginosis with Clindamycin. (Knowledge Centre for the Health Services at The Norwegian Institute of Public Health (NIPH), 2010).
58. Forney, L. J. et al. Comparison of Self-Collected and Physician-Collected Vaginal Swabs for Microbiome Analysis. J. Clin. Microbiol. 48, 1741-1748 (2010).
59. Parikh, H. I., Koparde, V. N., Bradley, S. P., Buck, G. A. & Sheth, N. U. MeFiT: merging and filtering tool for illumina paired-end reads for 16S rRNA amplicon sequencing. BMC Bioinformatics 17, 491 (2016).
60. Edgar, R. C., Haas, B. J., Clemente, J. C., Quince, C. & Knight, R. UCHIME improves sensitivity and speed of chimera detection. Bioinforma. Oxf. Engl. 27, 2194-2200 (2011).
61. Edgar, R. C. Search and clustering orders of magnitude faster than BLAST. Bioinforma. Oxf. Engl. 26, 2460-2461 (2010).
62. Yarza, P. et al. Update of the All-Species Living Tree Project based on 16S and 23S rRNA sequence analyses. Syst. Appl. Microbiol. 33, 291-299 (2010).
63. Chen, T. et al. The Human Oral Microbiome Database: a web accessible resource for investigating oral microbe taxonomic and genomic information. Database J. Biol. Databases Curation 2010, baq013 (2010).
64. Hartmann, M., Howes, C. G., Abarenkov, K., Mohn, W. W. & Nilsson, H. V-Xtractor: An open-source, high-throughput software tool to identify and extract hypervariable regions of small subunit (16 S/18 S) ribosomal RNA gene sequences. J. Microbiol. Methods 83, 250-3 (2010).
65. Lindgreen, S. AdapterRemoval: easy cleaning of next-generation sequencing reads. BMC Res. Notes 5, 337 (2012).
66. Koparde, V. N., Parikh, H. I., Bradley, S. P. & Sheth, N. U. MEEPTOOLS: a maximum expected error based FASTQ read filtering and trimming toolkit. Int. J. Comput. Biol. Drug Des. 10, 237-247 (2017).
67. Li, H. & Durbin, R. Fast and accurate long-read alignment with Burrows-Wheeler transform. Bioinforma. Oxf. Engl. 26, 589-595 (2010).
68. Alves, J. M. P. & Buck, G. A. Automated system for gene annotation and metabolic pathway reconstruction using general sequence databases. Chem. Biodivers. 4, 2593-2602 (2007).
69. Langille, M. G. I. et al. Predictive functional profiling of microbial communities using 16S rRNA marker gene sequences. Nat. Biotechnol. 31, 814-821 (2013).
70. Kaminski, J. et al. High-Specificity Targeted Functional Profiling in Microbial Communities with ShortBRED. PLOS Comput. Biol. 11, e1004557 (2015).
71. Altschul, S. F., Gish, W., Miller, W., Myers, E. W. & Lipman, D. J. Basic local alignment search tool. J. Mol. Biol. 215, 403-410 (1990).
72. Bankevich, A. et al. SPAdes: a new genome assembly algorithm and its applications to single-cell sequencing. J. Comput. Biol. J. Comput. Mol. Cell Biol. 19, 455-477 (2012).
73. Nurk, S., Meleshko, D., Korobeynikov, A. & Pevzner, P. A. metaSPAdes: a new versatile metagenomic assembler. Genome Res. 27, 824-834 (2017).
74. Langmead, B. & Salzberg, S. L. Fast gapped-read alignment with Bowtie 2. Nat. Methods 9, 357-359 (2012).
75. Lin, H.-H. & Liao, Y.-C. Accurate binning of metagenomic contigs via automated clustering sequences using information of genomic signatures and marker genes. Sci. Rep. 6, 24175 (2016).
76. Seemann, T. Prokka: rapid prokaryotic genome annotation. Bioinforma. Oxf. Engl. 30, 2068-2069 (2014).
77. Lowe, T. M. & Eddy, S. R. tRNAscan-SE: a program for improved detection of transfer RNA genes in genomic sequence. Nucleic Acids Res. 25, 955-964 (1997).
78. Lagesen, K. et al. RNAmmer: consistent and rapid annotation of ribosomal RNA genes. Nucleic Acids Res. 35, 3100-3108 (2007).
79. Aziz, R. K. et al. The RAST Server: rapid annotations using subsystems technology. BMC Genomics 9, 75 (2008).
80. Scholz, M. et al. Strain-level microbial epidemiology and population genomics from shotgun metagenomics. Nat. Methods 13, 435-438 (2016).
81. Brooks, J. P. et al. Changes in vaginal community state types reflect major shifts in the microbiome. Microb. Ecol. Health Dis. 28, 1303265 (2017).
82. Benjamini, Y. & Hochberg, Y. Controlling the False Discovery Rate: A Practical and Powerful Approach to Multiple Testing. J. R. Stat. Soc. Ser. B Methodol. 57, 289-300 (1995).
83. Lin, X. & Zhang, D. Inference in generalized additive mixed models by using smoothing splines. J. R. Stat. Soc. Ser. B Stat. Methodol. 61, 381-400 (1999).
84. Harville, D. Maximum likelihood approaches to variance component estimation and to related problems. J. Am. Stat. Assoc. 72, 320-338 (1977).
85. Parkhomenko, E., Tritchler, D. & Beyene, J. Sparse canonical correlation analysis with application to genomic data integration. Stat. Appl. Genet. Mol. Biol. 8, Article 1 (2009).
86. Witten, D. M., Tibshirani, R. & Hastie, T. A penalized matrix decomposition, with applications to sparse principal components and canonical correlation analysis. Biostatistics 10, 515-534 (2009).
87. González, I., Cao, K.-A. L., Davis, M. J. & Déjean, S. Visualising associations between paired ‘omics’ data sets. BioData Min. 5, 19 (2012).
88. Rohart, F., Gautier, B., Singh, A. & Lê Cao, K.-A. mixOmics: An R package for ‘omics feature selection and multiple data integration. PLoS Comput. Biol. 13, e1005752 (2017).
89. Ban, Y., An, L. & Jiang, H. Investigating microbial co-occurrence patterns based on metagenomic compositional data. Bioinforma. Oxf. Engl. 31, 3322-3329 (2015).
90. Bastian, M., Heymann, S. & Jacomy, M. Gephi: an open source software for exploring and manipulating networks. Int. AAAI Conf. Weblogs Soc. Media (2009).
91. Ng, A. Y. Feature Selection, L1 vs. L2 Regularization, and Rotational Invariance. in Proceedings of the Twenty-first International Conference on Machine Learning 78—(ACM, 2004). doi:10.1145/1015330.1015435
92. Pedregosa, F. et al. Scikit-learn: Machine learning in Python. J. Mach. Learn. Res. 12, 2825-2830 (2011).

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method for determining the risk of an adverse pregnancy outcome for a woman, comprising:
measuring an abundance of Saccharibacteria TM7-H1 and optionally one or more of BVAB1, Sneathia amnii, and Prevotella cluster 2 in a vaginal sample obtained from the woman, and
identifying the woman as having an increased risk for an adverse pregnancy outcome if the abundance is increased compared to corresponding standard control levels.

2. The method of claim 1, wherein the abundance of TM7-H1, BVAB1, Sneathia amnii, and Prevotella cluster 2 is measured.

3. The method of claim 1, further comprising measuring an abundance of one or more of: Dialister cluster 51, Prevotella amnii, Sneathia sanguinegens, Aerococcus christensenii, Clostridales BVAB2, Coriobacteriaceae OTU27, Dialister micraerophilus, Parvimonas OTU142, Megasphaera OTU70 type 1, and Lactobacillus crispatus cluster.

4. The method of claim 1, wherein the adverse pregnancy outcome is preterm birth (PTB).

5. The method of claim 1, wherein the woman is pregnant and the vaginal sample is obtained between 6 and 24 weeks of gestation.

6. The method of claim 1, wherein the woman is pregnant and the vaginal sample is obtained between 1 and 6 weeks of gestation.

7. The method of claim 1, wherein the vaginal sample is obtained prior to pregnancy.

8. The method of claim 1, wherein the woman is of African ancestry.

9. A method for determining the risk of an adverse pregnancy outcome for a woman and administering at least one prophylactic treatment to a woman identified as being at risk for an adverse pregnancy outcome, comprising:
i) measuring an abundance of TM7-H1 and optionally one or more of BVAB1, Sneathia amnii, and Prevotella cluster 2 in a vaginal sample obtained from the woman;
ii) identifying the woman as having an increased risk for an adverse pregnancy outcome if the abundance is increased compared to corresponding standard control levels; and
iii) administering the at least one prophylactic treatment for the adverse pregnancy outcome to the woman who is identified as having an increased risk for the adverse pregnancy outcome.

10. The method of claim 9, wherein the abundance of TM7-H1, BVAB1, Sneathia amnii, and Prevotella cluster 2 is measured.

11. The method of claim 9, further comprising measuring an abundance of one or more of: Dialister cluster 51, Prevotella amnii, Sneathia sanguinegens, Aerococcus christensenii, Clostridales BVAB2, Coriobacteriaceae OTU27, Dialister micraerophilus, Parvimonas OTU142, Megasphaera OTU70 type 1, and Lactobacillus crispatus cluster.

12. The method of claim 9, wherein the adverse pregnancy outcome is PTB.

13. The method of claim 9, wherein the woman is pregnant and the vaginal sample is obtained between 6 and 24 weeks of gestation.

14. The method of claim 9, wherein the woman is pregnant and the vaginal sample is obtained between 1 and 6 weeks of gestation.

15. The method of claim 9, wherein the vaginal sample is obtained prior to pregnancy.

16. The method of claim 9, wherein the at least one PTB prophylactic treatment is selected from the group consisting of antenatal corticosteroids, antibiotics, tocolytics, progesterone, cerclage application, products that modify the conditions of the female reproductive tract, prebiotics, probiotics, genetically engineered microbial or bacteriophage preparations, vaginal microbial transplants, and combinations thereof.

17. The method of claim 9, wherein the woman is of African ancestry.

18. A method of detecting an adverse pregnancy outcome risk, comprising:
obtaining a vaginal sample from a pregnant woman at a gestation of 6-24 weeks; and
detecting an abundance of TM7-H1, BVAB1, Sneathia amnii, and Prevotella cluster 2 in the vaginal sample.

19. A method of detecting an adverse pregnancy outcome risk, comprising:
obtaining a vaginal sample from a pregnant woman at a gestation of 1-6 weeks; and
detecting an abundance of TM7-H1, BVAB1, Sneathia amnii, and Prevotella cluster 2 in the vaginal sample.

20. A method of detecting an adverse pregnancy outcome risk, comprising:
obtaining a vaginal sample from a woman prior to pregnancy; and
detecting an abundance of TM7-H1, BVAB1, Sneathia amnii, and Prevotella cluster 2 in the vaginal sample.
